SEQUENCE LISTING (9 sequences)

SEQ ID NO: 1 - protein, 1620 aa, Homo sapiens [sequence omitted]

SEQ ID NO: 2 - protein, 563 aa, Homo sapiens
VYRRKHQELQAMQMELQSPEYKLSKLRTSTIMTDYNPNYCFAGKTSSISDLKEVPRKNNTLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEVSVRVPRGPAVEGGHVNMAFSQSNPPSELHRVHGSRNKPTSLWNPTYGSWFTEKPTKKNNPIAKKEPHERGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 3 - protein, 563 aa, Artificial Sequence
  Synthetic Construct
VYRRKHQELQAMQMELQSPEYKLSKLRTSTIMTDYNPNYCFAGKTSSISDLKEVPRKNNTLIRGLGHGAFGEVYEGQVSGMPNDPSPLQVAVRTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEVSVRVPRGPAVEGGHVNMAFSQSNPPSELHRVHGSRNKPTSLWNPTYGSWFTEKPTKKNNPIAKKEPHERGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 4 - protein, 302 aa, Artificial Sequence
  Synthetic Construct
CFAGKTSSISDLKEVPRKNNTLIRGLGHGAFGEVYEGQVSGMPNDPSPLQVAVRTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALP

SEQ ID NO: 5 - protein, 370 aa, Artificial Sequence
  Synthetic Construct
CFAGKTSSISDLKEVPRKNNTLIRGLGHGAFGEVYEGQVSGMPNDPSPLQVAVRTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEVSVRVP

SEQ ID NO: 6 - protein, 680 aa, Artificial Sequence
  Synthetic Construct
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVVYRRKHQELQAMQMELQSPEYKLSKLRTSTIMTDYNPNYCFAGKTSSISDLKEVPRKNNTLIRGLGHGAFGEVYEGQVSGMPNDPSPLQVAVRTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEVSVRVPRGPAVEGGHVNMAFSQSNPPSELHRVHGSRNKPTSLWNPTYGSWFTEKPTKKNNPIAKKEPHERGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 7 - protein, 419 aa, Artificial Sequence
  Synthetic Construct
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVCFAGKTSSISDLKEVPRKNNTLIRGLGHGAFGEVYEGQVSGMPNDPSPLQVAVRTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALP

SEQ ID NO: 8 - protein, 487 aa, Artificial Sequence
  Synthetic Construct
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVCFAGKTSSISDLKEVPRKNNTLIRGLGHGAFGEVYEGQVSGMPNDPSPLQVAVRTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEVSVRVP

SEQ ID NO: 9 - protein, 347 aa, Artificial Sequence
  Synthetic Construct
QELQAMQMELQSPEYKLSKLRTSTIMTDYNPNYCFAGKTSSISDLKEVPRKNITLIRGLGHGAFGEVYEGQVSGMPNDPSPLQVAVRTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEK

(19) United States
(12) Patent Application Publication
(10) Pub. No.: US 2018/0000906 A1
(43) Pub. Date: Jan. 4, 2018

(54) MULTILAMELLAR LIPID VESICLE COMPOSITIONS INCLUDING A CONJUGATED ANAPLASTIC LYMPHOMA KINASE (ALK) VARIANT AND USES THEREOF

(71) Applicant: Vedantra Phannaceuticals, Inc., Cambridge, MA (US)

(72) Inventors: Adrienne LI, Cambridge, MA (US); Jackson EBY, Arlington, MA (US); Peter C. DEMUTH, Medford, MA (US)

(73) Assignee: Vedantra Pharmaceuticals, Inc., Cambridge, MA (US)

(21) Appl. No.: 15/543,127

(22) PCT Filed: Jan. 15, 2016
(86) PCT No.: PCT/US2016/013635

§ 371 Date: Jul. 12, 2017

Related U.S. Application Data
(60) Provisional application No. 62/104,293, filed on Jan. 16, 2015.

(51) Int. Cl. A61K 38/45; C12N 9/78; C12N 9/12; C12N 9/10; A61K 31/4545; A61K 38/50; C07K 14/495; C07K 14/47; A61K 39/395; A61K 39/39; A61K 39/00; A61K 47/69; A61K 31/506

(52) U.S. Cl. CPC A61K 38/45; C12Y 305/0401; A61K 47/6911; C12Y 201/02003; C12N 9/78; C12N 9/12; C12N 9/1014; C12Y 207/10001; A61K 31/4545; A61K 31/506; C07K 14/495; C07K 14/47; A61K 39/3955; A61K 39/395; A61K 39/39; A61K 39/0011; A61K 38/50; A61K 2039/57; C07K 2319/10; C07K 2319/40

(57) ABSTRACT

The invention provides compositions including stabilized multilamellar lipid vesicles having crosslinked lipid bilayers (referred to herein as interbilayer-crosslinked multilamellar vesicles or ICMV) and including an ALK variant, pharmaceutical compositions containing vesicles (e.g., ICMV) including an ALK variant, and methods of treatment using such compositions. The invention provides compositions including stabilized multilamellar lipid vesicles with crosslinked lipid bilayers (e.g., an interbilayer-crosslinked multilamellar vesicle or ICMV) containing an Anaplastic lymphoma kinase (ALK) variant as an antigen that is associated with solid tumor cancers.

### BACKGROUND OF THE INVENTION

Anaplastic lymphoma kinase (ALK) is a receptor tyrosine kinase first identified in a chromosomal translocation associated with anaplastic large cell lymphomas (ALCL), a subset of T-cell non-Hodgkin lymphomas. Within ALCLs, nearly 70% of the cases carry the t(2;5)(p23;q35) chromosomal translocation that juxtaposes ALK locus to nucleophosmin (NPM) gene locus, generating a fusion protein of NPM and the cytoplasmic domain of ALK. Other ALK fusion proteins have been identified, including tropomyosin (TMP3), 5-Aminoimidazole-4-carboxamide ribonucleotide formyltransferase/IMP cyclohydrolase (ATIC), transforming growth factor (TGF), and echinoderm microtubule-associated protein-like 4 (EML4), in different types of solid tumors, such as non-small-cell lung cancers, neuroblastoma, rhabdomyosarcoma, neuroectodermal tumors, and glioblastomas. Data from human patients carrying ALK-positive ALCL show that the ALK protein is immunogenic; the elicited immune response involves CD8⁺ CTL cells, CD4⁺ T helper cells, and the production of anti-ALK antibodies, and the immune response could influence the outcome of the disease.

Available prophylactic vaccines often prevent but cannot cure existing cancers and ALK subunit vaccines to date have failed to produce essential cellular immunity. Prior cancer vaccines based on recombinant proteins avoided toxicity and anti-vector immunity associated with live vaccine (e.g., viral) vectors, but their immunogenicity was poor, particularly for CD8⁺ T-cell (CD8T) responses. Synthetic particles carrying antigens and adjuvant molecules have been developed to enhance subunit vaccines, but in general these materials have failed to elicit CD8T responses comparable to live vectors in preclinical animal models. There exists a need for novel and effective immunotherapy against cancers, such as solid tumor cancers.

### SUMMARY OF THE INVENTION

The invention provides compositions including stabilized multilamellar lipid vesicles with crosslinked lipid bilayers (e.g., an interbilayer-crosslinked multilamellar vesicle or ICMV) containing an Anaplastic lymphoma kinase (ALK) variant as an antigen that is associated with solid tumor cancers. When used to deliver antigen alone or in the presence of adjuvant, the vesicles of the invention form an extremely potent vaccine (e.g., a whole-protein vaccine), eliciting endogenous T-cell and antibody responses comparable to the strongest vaccine vectors. The invention also features pharmaceutical compositions containing vesicles (e.g., ICMV) including an ALK variant and methods of treatment using such compositions. In some embodiments, the vesicle (e.g., ICMV) including an ALK variant may be used in combination with one or more adjuvants and/or potentiating agents (e.g., an immunostimulatory or immunoinhibitory agent). In other embodiments, the vesicle (e.g., ICMV) including an ALK variant may be used in the absence of an adjuvant. In some embodiments, the ALK variant is a mutant or fragment of wild-type ALK. In other embodiments, the ALK variant further includes a NPM protein, a TMP3 protein, an ATIC protein, a TGF protein, or an EML4 protein.

In a first aspect, the invention features a composition including: (a) a multilamellar lipid vesicle having crosslinks between lipid bilayers; and (b) an anaplastic lymphoma kinase (ALK) variant. In some embodiments, the ALK variant is conjugated to a lipid.

In some embodiments, the composition further includes a nucleophosmin (NPM) protein or a fragment thereof. In some embodiments, the fragment of the NPM protein is an extracellular domain of the NPM protein. In certain embodiments, the NPM protein is fused to the ALK variant.

In some embodiments, the composition further includes a tropomyosin (TMP3) protein or a fragment thereof. In some embodiments, the fragment of the TMP3 protein is an extracellular domain of the TMP3 protein. In certain embodiments, the TMP3 protein is fused to the ALK variant.

In other embodiments, the composition further includes a 5-Aminoimidazole-4-carboxamide ribonucleotide formyltransferase/IMP cyclohydrolase (ATIC) protein or a fragment thereof. In some embodiments, the fragment of the ATIC protein is an extracellular domain of the ATIC protein. In certain embodiments, the ATIC protein is fused to the ALK variant.

In yet other embodiments, the composition further includes a transforming growth factor (TGF) protein or a fragment thereof. In some embodiments, the fragment of the TGF protein is an extracellular domain of the TGF protein. In certain embodiments, the TGF protein is fused to the ALK variant.

In still other embodiments, the composition further includes an echinoderm microtubule-associated protein-like 4 (EML4) protein or a fragment thereof. In some embodiments, the fragment of the EML4 protein is an extracellular domain of the EML4 protein. In certain embodiments, the EML4 protein is fused to the ALK variant.

In all of the aforementioned embodiments, at least two lipid bilayers in the multilamellar lipid vesicle are covalently crosslinked to each other through headgroups that react with covalent crosslinkers to form the covalent crosslinks between lipid bilayers.

In some embodiments, the lipid bilayers include anionic and/or neutral lipids. In other embodiments, the lipid bilayers include cationic lipids.

In all of the aforementioned embodiments, the composition may further include an adjuvant or a potentiating agent.

In some embodiments, the potentiating agent is a small molecule, which may be an anti-cancer agent (e.g., a tyrosine kinase inhibitor). In certain embodiments, the anti-cancer agent is Crizotinib. In certain embodiments, the anti-cancer agent is Ceritinib.

In other embodiments, the potentiating agent is an antibody, which may be an immunomodulatory agent. In some embodiments, the immunomodulatory agent is selected from the group consisting of an anti-PD-1 antibody, an anti-PD-L1 antibody, and an anti-CTLA-4 antibody. In yet other embodiments, the potentiating agent is an immunostimulatory agent, which may be a toll-like receptor (TLR) ligand.

In a second aspect, the invention features a pharmaceutical composition containing a therapeutically effective amount of the composition of the first aspect of the invention and one or more pharmaceutically acceptable carriers or excipients.

In a third aspect, the invention features a method of treatment including administering the pharmaceutical composition of the second aspect of the invention to a subject in need thereof.

In some embodiments, the pharmaceutical composition is administered without an adjuvant.

In some embodiments, the pharmaceutical composition is administered before or after administration of a potentiating agent. In other embodiments, the pharmaceutical composition is administered substantially simultaneously with a potentiating agent. The potentiating agent may be a small molecule, such as an anti-cancer agent. In some embodiments, the anti-cancer agent is a tyrosine kinase inhibitor (e.g., Crizotinib or Ceritinib).

In some embodiments, the subject has cancer. The cancer may be a solid tumor cancer or a cancer that expresses ALK or a portion thereof (an ALK⁺ cancer).

In some embodiments, the cancer is anaplastic large cell lymphoma, non-small-cell lung cancer, neuroblastoma, rhabdomyosarcoma, neuroectodermal cancer, glioblastoma, breast carcinoma, melanoma, inflammatory myofibroblastic tumor, soft tissue tumor, ALK expressing lymphoma, or ALK expressing lung, colon, or prostate carcinoma.

In other embodiments, the cancer is selected from bladder cancer, pancreatic cancer, lung cancer, liver cancer, ovarian cancer, colon cancer, stomach cancer, breast cancer, prostate cancer, renal cancer, testicular cancer, thyroid cancer, uterine cancer, rectal cancer, a cancer of the respiratory system, a cancer of the urinary system, oral cavity cancer, skin cancer, leukemia, sarcoma, carcinoma, basal cell carcinoma, non-Hodgkin's lymphoma, acute myeloid leukemia (AML), chronic lymphocytic leukemia (CLL), B-cells chronic lymphocytic leukemia (B-CLL), multiple myeloma (MM), erythroleukemia, renal cell carcinoma, astrocytoma, oligoastrocytoma, biliary tract cancer, choriocarcinoma, CNS cancer, larynx cancer, small cell lung cancer, adenocarcinoma, giant (or oat) cell carcinoma, and squamous cell carcinoma.

In some embodiments, the pharmaceutical composition is administered before or after surgery to remove at least some of a solid tumor in the solid tumor cancer.

In all of the aforementioned embodiments of the third aspect of the invention, the subject is a mammal. In some embodiments, the mammal is a human.

### DESCRIPTION OF THE DRAWINGS

FIG. 1A shows the sequence of wild-type full-length ALK (SEQ ID NO: 1; UniProt ID NO: B6D4Y2).

FIG. 1B shows the sequence of wild-type cytoplasmic domain of ALK (SEQ ID NO: 2).

FIG. 1C shows the sequence of cytoplasmic domain of ALK having K93R substitution (SEQ ID NO: 3).

FIG. 2 shows the overall T-cell response stimulated by an ALK variant (SEQ ID NO: 9) encapsulated in ICMVs.

### DETAILED DESCRIPTION OF THE INVENTION

The invention provides stabilized multilamellar lipid vesicles for use in, inter alia, delivery of an Anaplastic Lymphoma Kinase (ALK) variant. Prior vaccines based on recombinant proteins avoided toxicity and anti-vector immunity associated with live vaccine (e.g., viral) vectors, but their immunogenicity was poor, particularly for CD8⁺ T-cell (CD8T) responses. Synthetic particles carrying antigens and adjuvant molecules have been developed to enhance subunit vaccines, but in general these materials have failed to elicit CD8 T-cell responses comparable to live vectors in preclinical animal models. In contrast to these prior compositions and methods, the invention provides stabilized multilamellar vesicles, such as interbilayer-crosslinked multilamellar vesicles (ICMVs) formed by crosslinking headgroups of adjacent lipid bilayers within multilamellar vesicles in which an ALK variant has been covalently conjugated. These vesicles include protein antigens (e.g., conjugated protein antigens) and, optionally, immunostimulatory or immunoinhibitory molecules, but exhibit rapid release in the presence of endolysosomal lipases. The protein antigens and optional immunostimulatory or immunoinhibitory molecules may be present within the vesicle core, within the vesicle walls, or on an outer surface of the vesicle. When used to deliver antigen alone or in the presence of adjuvant, the vesicles of the invention form an extremely potent vaccine (e.g., a whole-protein vaccine), eliciting endogenous T-cell responses comparable to the strongest vaccine vectors.

The vesicles are stabilized by internal linking (e.g., crosslinking) of their lipid bilayers. The stabilized nature of these vesicles and covalent conjugation of an ALK variant allows them to incorporate higher amounts of the ALK variant and to retain such protein over a longer time period, as compared to simple liposomes or lipid coated nano- or microparticles. Their sustained release kinetics, particularly in the presence of serum, make them useful in in vivo delivery of an ALK variant for which a slow, steady and prolonged release is desirable or for which slow release in the extracellular environment but rapid release within cells is desirable. The invention therefore provides compositions including the aforementioned vesicles including an ALK variant, methods for their synthesis, and methods for their use. The present invention also incorporates by reference herein the disclosures of U.S. Pat. No. 8,747,869 (see, e.g., column 1, line 43 through column 5, line 40, and column 8, line 9 through column 35, line 69) and US Patent Publication No US20120177724 (see, e.g., p. 6, ¶ [0077] through p. 19, ¶ [0207]), which are directed to compositions, methods of synthesis, and methods of use of stabilized multilamellar vesicles.

### Stabilized Multilamellar Lipid Vesicles (MLV)

The invention provides MLV that are stabilized by linking adjacent (or apposed) lipid bilayers to one another and include ALK variants covalently conjugated to the vesicle (e.g., within the vesicle core, within the vesicle walls, or on an outer surface of the vesicle). In some embodiments, the invention provides vesicles that further include linking of two monolayers of a single bilayer. As used herein, a multilamellar vesicle is a nano- or microsphere having a shell that includes two or more concentrically arranged lipid bilayers. As used herein, adjacent or apposed lipid bilayers (or lipid bilayer surfaces) refer to bilayers or surfaces that are in close proximity to each other but that are otherwise distinct and typically physically separate. This term does not typically mean the relationship between the two monolayers of a single bilayer. In some embodiments, the multilamellar vesicle may include an ALK variant covalently conjugated to the vesicle (e.g., within the vesicle core, within the vesicle walls, or on an outer surface of the vesicle).

As used herein, “linking” means two entities stably bound to one another by any physiochemical means. Any linkage known to those of ordinary skill in the art may be employed including covalent or noncovalent linkage, although covalent linkage is preferred. In some important embodiments described herein, covalent linkage between lipid bilayers (e.g., adjacent or apposed lipid bilayers) in MLV is achieved through the use of crosslinkers and functionalized components of the lipid bilayer. The invention however contemplates that linking, including covalent linking, may be effected in other ways. As an example, the invention contemplates methods in which complementary reactive groups reside on components of adjacent bilayer surfaces and linkage between the bilayer surfaces is effected by reacting those groups to each other even in the absence of a crosslinker. Suitable complementary reactive groups are known in the art and described herein.

The interior of the vesicle is typically an aqueous environment, and it may include an ALK variant. In some instances, the vesicles do not include a solid core, such as a solid polymer core (e.g., a synthetic polymer core). Instead, as discussed above, they may have a fluid core including an ALK variant. The core may include monomers for polymerization into a hydrogel core in some instances. The vesicles may also be referred to herein as particles, including nano- or microparticles, although it is to be understood that such nano- or micro-particles have the attributes of the stabilized MLVs and interbilayer crosslinked multilamellar lipid vesicles (ICMVs) of the invention.

The vesicles may have a void volume at their core and/or they may include one or more ALK variants in their core and/or between adjacent (or apposing) lipid bilayers. The conjugated ALK variants may be covalently attached to functionalized lipids (e.g., maleimide functionalized lipids) through a reactive group (e.g., a thiol). Non-conjugated ALK variants may be included in the lipid solution during the synthesis process and in this manner are incorporated (e.g., by encapsulation) into the vesicles during synthesis. Adjuvants and/or potentiating agents may also be included in the lipid solution during the synthesis process and in this manner are incorporated (e.g., by encapsulation) into the vesicles during synthesis. Lipophilic molecules may also be incorporated directly into the lipid bilayers as the vesicles are formed or molecules with lipophilic tails may be anchored to the lipid bilayers during vesicle formation. The vesicles may be produced in the absence of harsh solvents, such as organic solvents, and as a result they may be able to encapsulate ALK variants.

The amount of an ALK variant in the vesicles may vary and may depend on the nature of the protein. In some embodiments, 10-500 μg of the ALK variant per mg of lipid may be incorporated into the vesicles of the invention. In some embodiments, the vesicles may include about 10 μg of the ALK variant, about 20 μg of the ALK variant, about 50 μg of the ALK variant, or 100 μg of the ALK variant, or about 150 μg of the ALK variant, or about 200 μg of the ALK variant, or about 250 μg of the ALK variant, or about 300 μg of the ALK variant, or about 325 μg of the ALK variant, or about 350 μg of the ALK variant, or about 375 μg of the ALK variant, or about 400 μg of the ALK variant, or about 500 μg of the ALK variant, per mg of lipid. In other embodiments, the vesicles may include 10-20 μg of the ALK variant per mg of lipid, or 15-60 μg of the ALK variant per mg of lipid, or 50-200 μg of the ALK variant per mg of lipid, or 100-300 μg of the ALK variant per mg of lipid, or 200-400 μg of the ALK variant per mg of lipid, or 300-500 μg of the ALK variant per mg of lipid.

The vesicles of the invention may also be characterized by their retention profiles. In some embodiments, the vesicles release an ALK variant at a rate of about 25% per week when placed in serum containing media (e.g., 10% serum) and maintained at 37° C. In some embodiments, the vesicles release about 25% of the ALK variant in the first week and up to about 90% after about 30 days under these conditions. In some embodiments, the vesicles maintain at least 80%, at least 85%, at least 90%, or at least 95% of their ALK variant when stored in buffer (such as HEPES+40% sorbitol) at 4° C. for at least 30 days (e.g., at least 60 days, at least 90 day, at least 120 days, at least 150 days, at least 180 days, at least 210 days).

The number of lipid bilayers in each vesicle may vary, with a typical range of at least 2 to about 50, or at least 2 to about 25, or at least 2 to about 15, or at least 2 to about 10, or at least 2 to about 5. The diameter of the vesicles may vary. In some instances, the vesicles will have a diameter ranging from about 100 to about 500 nm, including from about 125 to about 300 nm, including from about 150 to about 300 nm, including from about 175 to about 275 nm. In some instances, the diameter ranges from about 150 to about 250 nm. It will be understood that, in any preparation of vesicles, there will be heterogeneity between vesicles (e.g., relating to vesicle diameter, number of lipid bilayers, amount of loaded ALK variant).

As used herein, the vesicles of the invention may also be referred to as liposomes (e.g., stabilized multilamellar liposomes or, as discussed below, interbilayer crosslinked multilamellar liposomes). Accordingly, the use of the term “vesicles” is not intended to convey source or origin of the vesicles. The vesicles of the invention are synthetic vesicles (i.e., they are produced in vitro), as will be discussed in greater detail below.

The vesicles may be isolated, intending that they are physically separated in whole or in part from the environment in which they are synthesized. As an example, vesicles including an ALK variant may be separated in whole or in part from vesicles lacking the protein (i.e., empty vesicles), and may then be referred to as “isolated vesicles.” Separation may occur based on weight (or mass), density (including buoyant density), size, color, or other methods known in the art (e.g., where the cargo of the vesicle is detectable by its energy emission). Centrifugation can be used to separate vesicles of the invention from simple liposomes or MLVs of identical lipid composition that do not have crosslinked bilayers. For example, centrifugation at about 21,000 g for about 5 minutes is sufficient to separate the vesicles of the invention, which pellet, from these other particle types.

### Interbilayer Crosslinked Multilamellar Lipid Vesicles

An example of the stabilized MLV of the invention is the interbilayer crosslinked multilamellar (lipid) vesicles (ICMV). Like the stabilized MLV described above, ICMV are nano- or microspheres having a shell that includes of two or more concentrically arranged lipid bilayers that are conjugated to each other as described herein. The number of lipid bilayers in the stabilized multilamellar vesicles, including the ICMV, may vary from about 2-30 (e.g., 2-15, 5-20, 10-30). Accordingly, in various embodiments, the number of layers may be 2, 3, 4, 5, 6, 7, 8, 9, 10, 11, 12, 13, 14, 15 or more. The bilayers are typically composed of lipids having hydrophilic heads and hydrophobic tails that are arranged in a manner similar to a cell membrane (i.e., with the hydrophilic heads exposed to typically an aqueous environment and the hydrophobic tails buried in the bilayer).

The ICMV are stabilized via crosslinks between their lipid bilayers, and they are therefore referred to as “interbilayer crosslinked” MLV. As used herein, this means that at least two lipid bilayers in the shell of the vesicle are crosslinked to each other. The crosslinked bilayers are typically those that are apposed or adjacent to each other. Most or all of the lipid bilayers in the shell may be crosslinked to their apposing lipid bilayer in the shell. There may be one or more crosslinks between lipid bilayers. Typically, there will be numerous crosslinks between lipid bilayers. The arrangement and positioning of such crosslinks may be random or non-random. The degree of crosslinks (and thus the resultant stability of the vesicles) will depend upon the proportion of functionalized lipids (or other lipid bilayer components) used to make the vesicles and the crosslinking conditions (including, for example, time of incubation of the vesicles with a crosslinker). It will be understood that the higher the proportion of functionalized lipids (or other lipid bilayer components) in the vesicles, the more crosslinks that will be formed, all other factors and parameters being equal. Similarly, the more favorable the conditions towards crosslinking, the greater degree of crosslinking that will be achieved.

### Synthesis Methods

An exemplary synthesis method is as follows: Lipids and optionally other bilayer components are combined to form a homogenous mixture. This may occur through a drying step in which the lipids are dried to form a lipid film. The lipids are then combined (e.g., rehydrated) with an aqueous solvent. The aqueous solvent may have a pH in the range of about 6 to about 8, including a pH of about 7. Buffers compatible with vesicle fusion are used, typically with low concentrations of salt (e.g., a 10 mM bis-tris propane (BTP) pH 7.0 buffer). The nature of the buffer may impact the length of the incubation. For example, a buffer such as HEPES may require a longer incubation time as compared to a buffer such as BTP. If the buffer is HEPES, then the incubation times may be about 6-24 hours, or 8-16 hours, or 10-12 hours. If the buffer is BTP, then the incubation times may be shorter including 1-4 hours, or 1-2 hours. Accordingly a variety of aqueous buffers may be used provided that a sufficient incubation time is also used. This step may also include the presence of the ALK variant to be incorporated into the vesicles. The resultant liposomes may then be fluidized, for example, on a Microfluidics LV-1 with 1-4 fluidizer passes (e.g., 1-2 passes) under a pressure of between 10000-40000 psi (e.g., 25000-35000) to collect between 1-13 mL (e.g., 3-4 mL) from the fluidizer. Alternatively, the liposomes may be incubated with one or more divalent cations in order to fuse them into multilamellar vesicles. Suitable divalent cations include Mg²⁺, Ca²⁺, Ba²⁺, or Sr²⁺. Multivalent or polymeric cations could also be employed for vesicle fusion. Vesicle fusion could also be achieved via the mixing of cationic vesicles with divalent or higher valency anions; an example would be fusion of cationic liposomes with DNA oligonucleotides or DNA plasmids. This may be done under agitation such as sonication or vortexing. If the liposomes were made in the presence of an ALK variant (e.g., a functionalized ALK variant), the MLVs will include the ALK variant in their core, between the concentrically arranged lipid bilayers, and/or on an outer surface. The invention contemplates fusion of liposomes carrying different ALK variants to form MLVs that include such ALK variants.

The resultant MLVs are then incubated with an ALK variant (e.g., a functionalized ALK variant prepared in parallel) under conditions suitable for conjugation of the ALK variant to a lipid. Conjugation may be carried out by incubating for 0.5-48 hours (e.g., 6-24 hours) at room temperature or elevated temperature (e.g., 37° C.) depending on the conjugation method used. Following conjugation of an ALK variant, the MLVs are incubated (e.g., for 0.5-6 hours preferably 1-2 hours) with a crosslinker, such as a membrane-permeable crosslinker, and optionally a divalent cation source such as calcium chloride (CaCl₂) (e.g., about 100-1000 μL CaCl₂ per mL total volume, preferably 150-300 μL/mL). The amount of crosslinker will vary depending on the nature of the reactive groups being linked together, for example, about 10-100 μL dithiothrietol (DTT) per mL lipids (e.g., 20-30 μL/mL) may be used if the functionalized lipids are maleimide functionalized lipids. As stated herein, the nature of the crosslinker will vary depending on the nature of the reactive groups being linked together. For example, a dithiol-containing crosslinker such as DTT or (1,4-Di-[3′-(2′-pyridyldithio)-propionamido]butane) may be used to crosslink MLVs composed of maleimide functionalized lipids (or other functionalized lipid bilayer components), or diazide crosslinkers could be used to crosslink alkyne headgroup lipids via “click” chemistry. These various incubations are all carried out under aqueous conditions at a pH in the range of about 6 to about 8, or about 6.5 to about 7.5, or at about 7. The crosslinking step may be performed at room temperature (e.g., 20-25° C.) or at an elevated temperature including for example up to or higher than 37° C.

The resultant crosslinked vesicles may then be collected (e.g., by centrifugation or other pelleting means), washed and then optionally PEGylated on their outermost or external surface (e.g., as used herein, the vesicles may be referred to “surface-PEGylated” or “surface-conjugated” to PEG) by incubation with a thiol-PEG. The PEG may be of any size, including but not limited to 0.1-10 kDa, 0.5-5 kDa, or 1-3 kDa, such as a 2 kDa PEG-SH. The incubation period may range from about 10 minutes to 6 hours (e.g., 1 to 3 hours), although it may be shorter or longer depending on other conditions such as temperature and concentration. The PEGylation step may be performed at room temperature (e.g., 20-25° C.) or at an elevated temperature including for example up to or higher than 37° C. The vesicles then may be collected (e.g., by centrifugation or other pelleting means) and washed with water or other aqueous buffer. Centrifugation may be performed at 1000-22000×g (e.g., 3000-21000×g) for about 1 to 30 minutes (e.g., 5 to 15 minutes).

The vesicles may then be extruded through a 0.2 μm membrane one to thirty times (e.g., five to fifteen times such as nine times).

The vesicles may be stored at 4° C. in a buffered solution such as but not limited to HEPES solutions containing sucrose, free PEG, polysorbate 20 (PS-20), or sorbitol (e.g., 1-80% w/v sorbitol, 35-45% w/v sorbitol, 40% w/v sorbitol) or they may be lyophilized in the presence of suitable cryopreservants and then stored at −20° C. Suitable cryopreservants include those that include sucrose (e.g., a 1-5% sucrose, and preferably about 3% sucrose solution).

Crosslinking could also be achieved by coupling between a reactive group in one bilayer with a complementary reactive group in the adjacent bilayer. For example, fused vesicles containing succinimidyl ester-functionalized lipid (A) headgroups and primary-amine-containing (B) headgroups could achieve crosslinking by in situ reaction between the A and B lipids of adjacent bilayers. A variety of other complementary functionalized lipids familiar to those skilled in the art could be employed in a similar manner.

The molar ratio of functionalized lipid (or other functionalized component of the lipid bilayer) to crosslinker may vary depending on the conditions. In some instances, it may range from about 1 to about 5. In some embodiments, a molar ratio of 2 is sufficient (i.e., the molar ratio of functionalized lipid (or component) to crosslinker is 2:1). For example, a 2:1 molar ratio of maleimide functionalized lipid to DTT may be used to crosslink the lipid bilayers of the vesicles. The incubation time may range from 1 hour to 24 hours, from 2-18 hours, from 2 to 12 hours, or from 2 to 6 hours. In some instances, it may be about 2 hours. In other instances, it may be overnight (e.g., about 12 hours).

The molar % of the functionalized lipid in the vesicles may range from 1% to 100% or from about 10% to about 60% in some instances, or from about 25% to about 55% in some instances. In some instances, the molar % of the functionalized lipid in the vesicles is typically at least 10%, preferably at least 15%, more preferably at least 20%, and even more preferably at least 25%.

Conversely, the non-functionalized lipids may be present at about 0% to 99% as a molar %. More typically, the non-functionalized lipids may be present at about 40%-75% or 40% to 60% as a molar %.

In one embodiment, the vesicles are synthesized using DOPC, DOPG, and maleimide-functionalized DSPE. The ratio of these lipids to each other may vary. The molar % of DOPC may range from 1-50%, the molar % of DOPG may range from 1-50%, and the molar % of the maleimide functionalized lipid may range from 1-80%. In another embodiment, no DOPG is used. Some embodiments of the invention provide vesicles having a DOPC:DOPG:maleimide functionalized lipid ratio of 40:10:50. Some embodiments provide vesicles having a DOPC:DOPG:maleimide functionalized lipid ratio of 60:15:25. Some embodiments provide vesicles composed of DOPG and a maleimide functionalized lipid.

### Lipids

The vesicles are composed of one or more lipids. The type, number and ratio of lipids may vary with the proviso that collectively they form spherical bilayers (i.e., vesicles). The lipids may be isolated from a naturally occurring source or they may be synthesized apart from any naturally occurring source.

At least one (or some) of the lipids is/are amphipathic lipids, defined as having a hydrophilic and a hydrophobic portion (typically a hydrophilic head and a hydrophobic tail). The hydrophobic portion typically orients into a hydrophobic phase (e.g., within the bilayer), while the hydrophilic portion typically orients toward the aqueous phase (e.g., outside the bilayer, and possibly between adjacent apposed bilayer surfaces). The hydrophilic portion may include polar or charged groups such as carbohydrates, phosphate, carboxylic, sulfato, amino, sulfhydryl, nitro, and hydroxy groups. The hydrophobic portion may include apolar groups that include without limitation long chain saturated and unsaturated aliphatic hydrocarbon groups and groups substituted by one or more aromatic, cyclo-aliphatic or heterocyclic group(s). Examples of amphipathic compounds include, but are not limited to, phospholipids, aminolipids and sphingolipids.

In some embodiments, the lipids are phospholipids. Phospholipids include without limitation phosphatidylcholine, phosphatidylethanolamine, phosphatidylglycerol, phosphatidylinositol, and phosphatidylserine. It is to be understood that other lipid membrane components, such as cholesterol, sphingomyelin, and cardiolipin may be used.

The lipids may be cationic, anionic, and/or neutral (including zwitterionic and polar) lipids. In some embodiments, the lipids are anionic and/or neutral (e.g., anionic or neutral phospholipids). Neutral lipids exist in an uncharged or neutral zwitterionic form at a selected pH. At physiological pH, such lipids include, for example, dioleoylphosphatidylglycerol (DOPG), diacylphosphatidylcholine, diacylphosphatidylethanolamine, ceramide, sphingomyelin, cephalin, cholesterol, cerebrosides and diacylglycerols. Examples of zwitterionic lipids include without limitation dioleoylphosphatidylcholine (DOPC), dimyristoylphosphatidylcholine (DMPC), and dioleoylphosphatidylserine (DOPS). An anionic lipid is a lipid that is negatively charged at physiological pH. These lipids include without limitation phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

Collectively, anionic and neutral lipids are referred to herein as non-cationic lipids. Such lipids may contain phosphorus but they are not so limited. Examples of non-cationic lipids include lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol.

Additional nonphosphorous containing lipids include stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, diacylphosphatidylcholine, diacylphosphatidylethanolamine, ceramide, sphingomyelin, cephalin, and cerebrosides. Lipids such as lysophosphatidylcholine and lysophosphatidylethanolamine may be used in some instances. Noncationic lipids also include polyethylene glycol-based polymers such as PEG 2000, PEG 5000 and polyethylene glycol conjugated to phospholipids or to ceramides (referred to as PEG-Cer).

In some instances, modified forms of lipids may be used including forms modified with detectable labels such as fluorophores. In some instances, the lipid is a lipid analog that emits signal (e.g., a fluorescent signal). Examples include without limitation 1,1′-dioctadecyl-3,3,3′,3′-tetramethylindotricarbocyanine iodide (Di R) and 1,1′-dioctadecyl-3,3,3′,3′-tetramethylindodicarbocyanine (DiD).

Preferably, the lipids are biodegradable in order to allow release of encapsulated ALK variant in vivo and/or in vitro. Biodegradable lipids include but are not limited to 1,2-dioleoyl-sn-glycero-3-phosphocholine (dioleoyl-phosphocholine, DOPC), anionic 1,2-di-(9Z-octadecenoyl)-sn-glycero-3-phospho-(1′-rac-glycerol) (dioleoyl-phosphoglycerol, DOPG), and 1,2-distearoyl-sn-glycero-3-phosphoethanolamine(distearoyl-phosphoethanol-amine, DSPE). Non-lipid membrane components such as cholesterol may also be incorporated.

### Functionalized Lipids or Bilayer Components

At least one component of the lipid bilayer must be functionalized (or reactive). As used herein, a functionalized component is a component that includes a reactive group that can be used to crosslink adjacent bilayers of the multilamellar vesicle. The bilayer component may be modified to include the reactive group.

One or more of the lipids used in the synthesis of the vesicles may be functionalized lipids. As used herein, a functionalized lipid is a lipid having a reactive group that can be used to crosslink adjacent bilayers of the multilamellar vesicle. In some embodiments, the reactive group is one that will react with a crosslinker (or other moiety) to form crosslinks between such functionalized lipids (and thus between lipid bilayers in the vesicle). The reactive group may be located anywhere on the lipid that allows it to contact a crosslinker and be crosslinked to another lipid in an adjacent apposed bilayer. In some embodiments, it is in the head group of the lipid, including for example a phospholipid. An example of a reactive group is a maleimide group. Maleimide groups may be crosslinked to each other in the presence of dithiol crosslinkers such as but not limited to dithiolthrietol (DTT). An example of a functionalized lipid is 1,2-dioleoyl-sn-glycero-3-phosphoethanolamine-N-[4-(p-maleimidop-henyl) butyramide, referred to herein as MPB. Another example of a functionalized lipid is 1,2-distearoyl-sn-glycero-3-phosphoethanolamine-N-[maleimide(polyethylene glycol)2000] (also referred to as maleimide-PEG 2k-PE). Another example of a functionalized lipid is dioleoyl-phosphatidylethanolamine 4-(N-maleimidomethyl)-cyclohexane-1-carboxylate (DOPE-mal).

It is to be understood that the invention contemplates the use of other functionalized lipids, other functionalized lipid bilayer components, other reactive groups, and other crosslinkers. In addition to the maleimide groups, other examples of reactive groups include but are not limited to other thiol reactive groups, amino groups such as primary and secondary amines, carboxyl groups, hydroxyl groups, aldehyde groups, alkyne groups, azide groups, carbonyls, haloacetyl (e.g., iodoacetyl) groups, imidoester groups, N-hydroxysuccinimide esters, sulfhydryl groups, and pyridyl disulfide groups.

Functionalized and non-functionalized lipids are available from a number of commercial sources including Avanti Polar Lipids (Alabaster, Ala.).

It is to be understood that the invention contemplates various ways to link adjacent bilayers in the multilamellar vesicles to each other. In some instances, crosslinkers are used to effect linkage between adjacent bilayers. The invention however is not so limited.

As an example, vesicles may be formed using click chemistry. An exemplary synthesis method uses alkyne-modified lipids and alkyne-azide chemistry, as follows. Alkyne-modified lipids may be made by mixing the lipids such as 1,2-dioleoyl-sn-glycero-3-phosphoethanolamine (DOPE) with N-hydroxysuccinimide ester of propiolic acid and Et₃N in CDCl₃. The reaction may be monitored by NMR. After the reaction is completed, the organic solution may be washed with 5% Na₂CO₃, 1% HCl and brine, dried under Na₂SO₄ and evaporated, and alkyne-modified DOPE weighed. Lipid film with DOPC and alkyne-DOPE in 1:1 molar ratio may be prepared, hydrated, fluidized, and induced to fuse with Mg²⁺ as described previously. MLVs with alkyne-functionalized lipids may be incubated with CuSO₄, copper wire, and 1,14-diazido-3,6,9,12-tetraoxatetradecane for 24 hours at room temperature. Particles may be isolated by centrifugation.

### Crosslinkers

The crosslinker may be a homobifunctional crosslinker or a heterobifunctional crosslinker, depending upon the nature of reactive groups in the lipid bilayers that are being linked to each other. The terms “crosslinker” and “crosslinking agent” are used interchangeably herein. Homobifunctional crosslinkers have two identical reactive groups. Heterobifunctional crosslinkers have two different reactive groups.

In one instance, bilayers (e.g., adjacent bilayers) are crosslinked to each other using the same functionalized lipid (or other bilayer component) and a crosslinker (such as a homobifunctional crosslinker). In another instance, bilayers (e.g., adjacent bilayers) are crosslinked to each other using different functionalized lipids (or other bilayer components) and a crosslinker (such as a heterobifunctional crosslinker).

Various types of commercially available crosslinkers are reactive with one or more of the following groups: maleimides, primary amines, secondary amines, sulfhydryls, carboxyls, carbonyls and carbohydrates. Examples of amine-specific crosslinkers are bis(sulfosuccinimidyl) suberate, bis[2-(succinimidooxycarbonyloxy)ethyl]sulfone, disuccinimidyl suberate, disuccinimidyl tartarate, dimethyl adipimate.2HCl, dimethyl pimelimidate.2HCl, dimethyl suberimidate.2HCl, and ethylene glycolbis-[succinimidyl-[succinate]]. Crosslinkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Crosslinkers preferentially reactive with carbohydrates include azidobenzoyl hydrazine. Crosslinkers preferentially reactive with carboxyl groups include 4-[p-azidosalicylamido]butylamine. Dithiol crosslinkers such as dithiolthietol (DTT), 1,4-di-[3′-(2′-pyridyldithio)-propionamido]butane (DPDPB), and in some instances thiol containing polymers such as (PEG)-SH2 can be used to crosslink maleimide reactive groups.

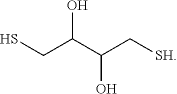

The structure of DTT is:

The structure of DPDPB is:

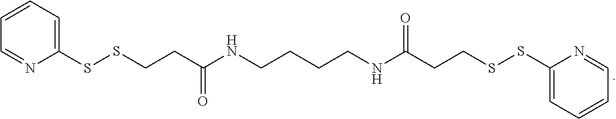 [SMILES: O=C(CCSSc1ccccn1)NCCCCNC(=O)CCSSc1ccccn1]

Crosslinkers reactive with alkyne groups include diazides, such as 1,14-Diazido-3,6,9,12-Tetraoxatetradecane, and other groups compatible with “click” chemistry.

Heterobifunctional crosslinkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional cross-linkers that react with carboxyl and amine groups include 1-ethyl-3-[3-dimethylaminopropyl]-carbodiimide hydrochloride. Heterobifunctional crosslinkers that react with carbohydrates and sulfhydryls include 4-[N-maleimidomethyl]-cyclohexane-1-carboxylhydrazide.2HCl, 4-(4-N-maleimidophenyl)-butyric acid hydrazide.2HCl, and 3-[2-pyridyldithio]propionyl hydrazide. Other crosslinkers are bis-[.beta.-4-azidosalicylamido)ethyl]disulfide and glutaraldehyde.

Crosslinkers may be membrane permeable (or lipid soluble) so that they may diffuse through one or more bilayers of the MLVs to effect crosslinking between various layers (e.g., adjacent layers). Any weakly polar/uncharged bifunctional or heterobifunctional small molecule may be an effective membrane permeable crosslinker, particularly if such a molecule includes a reactive group such as but not limited to maleimides, succinimidyl esters, azides, and thiols. Examples of membrane permeable crosslinkers include but are not limited to DTT and 1,4-di-[3′-(2′-pyridyldithio)-propionamido]butane (DPDPB).

### Conjugation of an Anaplastic Lymphoma Kinase (ALK) Variant

In the present invention, the vesicles of the invention include an ALK variant conjugated to a lipid of the vesicle. As used herein, “conjugated” refers to covalent attachment of the ALK variant to the lipid. The ALK variant may be covalently attached to the lipid by reaction of complementary reactive groups on each species. The ALK variant and/or lipid may be functionalized to contain the complementary reactive groups or the reactive groups may be a group already present in the ALK variant or lipid. For example, the ALK variant may include or be functionalized to include a thiol group and a covalent linkage formed by reaction with lipid functionalized to include a maleimide group. Alternatively, the reactive group on the ALK variant may be an amine or carboxylic acid and the covalent attachment to the lipid could be an amide bond formed by reaction with an amine or carboxylic acid of the lipid. The ALK variant may be conjugated to the lipid prior to vesicle (e.g., ICMV) synthesis, and therefore may be encapsulated within the vesicle, between the lipid bilayers of the vesicle, or present on the outer surface of the vesicle.

Reactive groups to be used to conjugate the ALK variant to the lipid may be the same as those used to crosslink the bilayers, in which case no additional functionalized lipids (or other functionalized components) are required. As an example, if the vesicles (e.g., ICMVs) include maleimide functionalized lipids, then the functionalized ALK variant may be thiol-functionalized ALK variant. Alternatively, the reactive groups used to stabilize the vesicles may be different from those used to conjugate the ALK variant to the lipid. Those of ordinary skill in the art will appreciate that other modified versions of ALK variant may be used depending on the nature of the reactive group in the functionalized lipid (or component) in the lipid bilayer of the vesicles. Suitable reactive groups include without limitation amino groups such as primary and secondary amines, carboxyl groups, sulfhydryl groups, hydroxyl groups, aldehyde groups, azide groups, carbonyls, maleimide groups, haloacetyl (e.g., iodoacetyl) groups, imidoester groups, N-hydroxysuccinimide esters, and pyridyl disulfide groups.

### Functionalized ALK Variant

An ALK variant may be functionalized or reactive. As used herein, a functionalized ALK variant is an ALK variant that includes a reactive group that can be used to conjugate the ALK variant to a lipid (e.g., a functionalized lipid). The ALK variant may be modified to include the reactive group.

An ALK variant used in the synthesis of the compositions of the invention may be functionalized ALK variant. In some embodiments, the reactive group is one that will react to form a covalent attachment to a lipid. The reactive group may be located anywhere on the ALK variant that allows it to be conjugated to a lipid. An example of a reactive group is a thiol group.

It is to be understood that the invention contemplates the use of other functionalized proteins and other reactive groups. In addition to the thiol group, other examples of reactive groups include but are not limited to other thiol reactive groups, amino groups such as primary and secondary amines, carboxyl groups, hydroxyl groups, aldehyde groups, alkyne groups, azide groups, carbonyls, haloacetyl (e.g., iodoacetyl) groups, imidoester groups, N-hydroxysuccinimide esters, sulfhydryl groups, and pyridyl disulfide groups.

An average of 1-2 molecules of added reactive group (e.g., a thiol) per ALK variant molecule is desirable for efficient conjugation of the ALK variant to the lipid. However, for some ALK variants, addition of more reactive groups (e.g., a thiol molecule) per ALK variant molecule may result in increased conjugation. As such, addition of 2, 3, 4, 5, or more reactive groups (e.g., thiols) per ALK variant molecule is encompassed by the invention.

As an example, ALK variants may be conjugated to lipids by reacting a thiol-functionalized ALK variant with a maleimide functionalized lipid. Thiol-functionalized ALK variants may be prepared using methods known in the art, e.g., 2-iminothiolane-HCl (Traut's reagent), N-succinimidyl S-acetylthioacetate hydrochloride (SATA), or N-Succinimidyl S-acetyl(thiotetraethylene glycol). For example, treatment of an ALK variant containing a primary amine with 10, 20, 30, 40, 50, 60, 70, 80, or more molar equivalents of Traut's reagent at room temperature (e.g., for 1 hour) provides thiol-functionalized ALK variants. An average of 1-2 molecules of added thiol per ALK variant molecule is desirable for efficient conjugation. However, for some ALK variants, addition of more thiol molecules per ALK variant molecule may result in increased conjugation. As such, addition of 2, 3, 4, 5, or more thiol molecule per ALK variant molecule is encompassed by the invention.

### Anaplastic Lymphoma Kinase (ALK) Variant

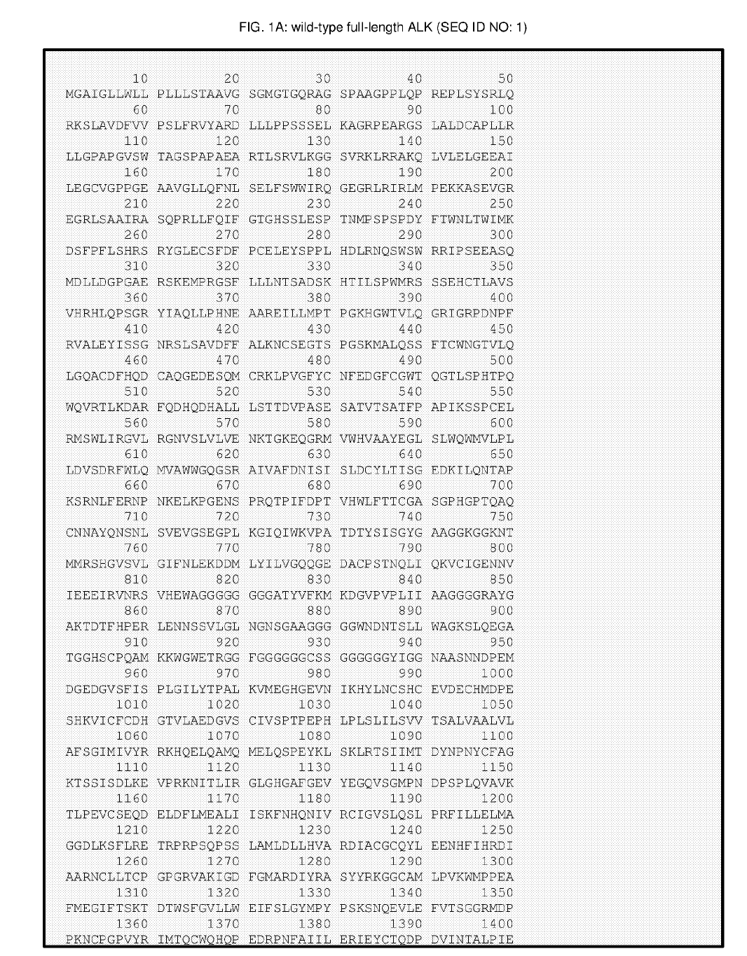

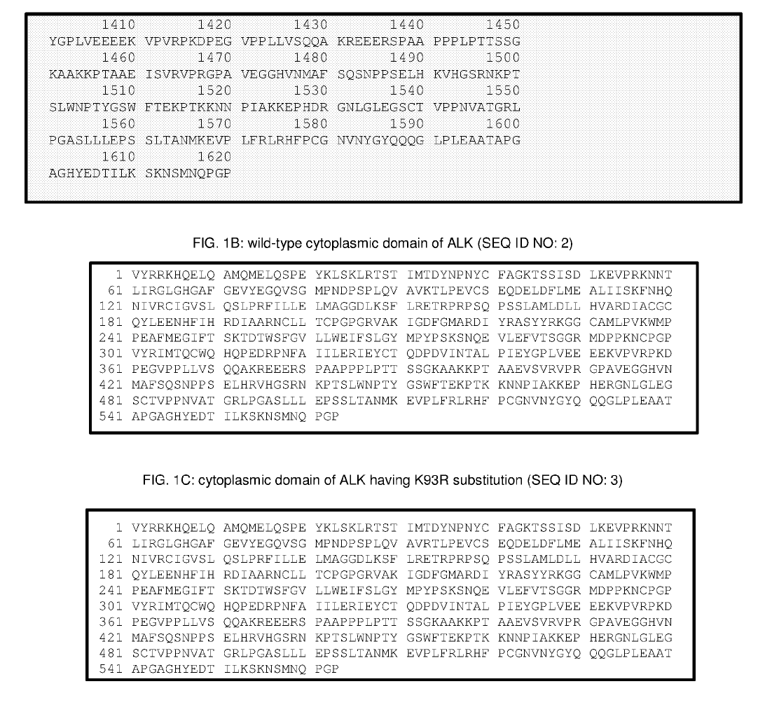

As used herein, ALK variant refers to a polypeptide containing a mutant or fragment of wild-type full-length ALK (SEQ ID NO: 1; FIG. 1A), where the fragment is at least 250, 275, 300, 325, or 350 amino acids in length. In some embodiments, an ALK variant has at least 80% sequence identity (e.g., at least 85%, 90%, 95%, 97%, or 100% sequence identity over the full length of the ALK variant sequence) to the ALK sequence (SEQ ID NO: 1; FIG. 1A). In some embodiments, an ALK variant has at least 80% sequence identity (e.g., at least 85%, 90%, 95%, 97%, or 100% sequence identity over the full length of the ALK variant sequence) to the sequence of the wild-type cytoplasmic domain of ALK (SEQ ID NO: 2; FIG. 1B). In some embodiments, an ALK variant has at least 80% sequence identity (e.g., at least 85%, 90%, 95%, 97%, or 100% sequence identity over the full length of the ALK variant sequence) to the sequence having amino acids 40-409 or 40-341 of SEQ ID NO: 2. In some embodiments, an ALK variant has at least 80% sequence identity (e.g., at least 85%, 90%, 95%, 97%, or 100% sequence identity over the full length of the ALK variant sequence) to the ALK variant having the sequence of SEQ ID NO: 9. In some embodiments, an ALK variant contains amino acid substitution K93R relative to the wild-type cytoplasmic domain of ALK that renders the tyrosine kinase domain of ALK inactive and ALK variant non-oncogenic. In certain embodiments, the ALK variant consists of the sequence of the cytoplasmic domain of ALK having K93R substitution (SEQ ID NO: 3 (see Example 1); FIG. 10), or amino acids 40-409 or 40-341 of SEQ ID NO: 3.

In some embodiments, an ALK variant is part of a fusion protein that includes a nucleophosmin (NPM) protein, a tropomyosin (TMP3) protein, a 5-Aminoimidazole-4-carboxamide ribonucleotide formyltransferase/IMP cyclohydrolase (ATIC) protein, a transforming growth factor (TGF) protein, an echinoderm microtubule-associated protein-like 4 (EML4) protein, or a fragment thereof (i.e., an extracellular domain thereof). Each of the NPM protein, TMP3 protein, ATIC protein, TGF protein, EML4 protein, and a fragment thereof (i.e., an extracellular domain thereof) may be attached to the N- or C-terminus of the ALK variant.

### Adjuvants

Adjuvant refers to one or more substances that cause stimulation of the immune system. In this context, an adjuvant is used to enhance an immune response to one or more antigens (e.g., an ALK variant). An adjuvant may be administered to a subject before, in combination with, or after administration of the antigens (e.g., an ALK variant), an ALK variant within a vesicle (e.g., ICMV).

The adjuvant may be without limitation lipids (e.g., monophosphoryl lipid A (MPLA)), alum (e.g., aluminum hydroxide, aluminum phosphate); saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), Montanide IMS (e.g., IMS1312, water-based nanoparticles combined with a soluble immunostimulant, Seppic), and CDNs (cyclic di-nucleotides).

Adjuvants may be toll-like receptor (TLR) ligands. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

Liposome formulations may confer adjuvant effects, and therefore liposome adjuvants are included according to the invention. Adjuvants can facilitate uptake of the molecules in the immunogenic composition by antigen presenting cells (APCs), such as dendritic cells, and activate these cells. An adjuvant's ability to increase an immune response is manifested by an increase in immune mediated protection. Enhancement of humoral immunity can be determined by, for example, an increase in the titer of antibody raised to the antigen. Enhancement of cellular immunity can be measured by, for example, a positive skin test, cytotoxic T-cell assay, ELISPOT assay for IFN-gamma or IL-2.

### Potentiating Agents

A potentiating agent refers to an agent, such as a molecule (e.g., a small, organic molecule), peptide, protein (e.g., an antibody or an antigen), or a drug that can enhance or augment the effectiveness of a therapeutic compound or composition in treating a disease. The type of potentiating agent used together with the therapeutic compound or composition depends on the specific disease being treated. Potentiating agents include, but are not limited to, anticancer agents, immunostimulatory agents, immunoinhibitory agents, cancer antigens, anti-infective agents, anti-viral agents, and anti-fungal agents. For example, the composition described herein containing a vesicle (e.g., an ICMV) including an ALK variant for the treatment of cancer (e.g., a solid tumor cancer) may be used in combination with an anticancer agent as the potentiating agent.

Anticancer Agents

As used herein, an anti-cancer agent is an agent that at least partially inhibits the development or progression of a cancer, including inhibiting in whole or in part symptoms associated with the cancer even if only for the short term. Several anti-cancer agents can be categorized as DNA damaging agents and these include topoisomerase inhibitors (e.g., etoposide, ramptothecin, topotecan, teniposide, mitoxantrone), DNA alkylating agents (e.g., cisplatin, mechlorethamine, cyclophosphamide, ifosfamide, melphalan, chorambucil, busulfan, thiotepa, carmustine, lomustine, carboplatin, dacarbazine, procarbazine), DNA strand break inducing agents (e.g., bleomycin, doxorubicin, daunorubicin, idarubicin, mitomycin C), anti-microtubule agents (e.g., vincristine, vinblastine), anti-metabolic agents (e.g., cytarabine, methotrexate, hydroxyurea, 5-fluorouracil, floxuridine, 6-thioguanine, 6-mercaptopurine, fludarabine, pentostatin, chlorodeoxyadenosine), anthracyclines, vinca alkaloids, or epipodophyllotoxins.

Examples of anti-cancer agents include without limitation Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Bortezomib (VELCADE); Brequinar Sodium; Bropirimine; Busulfan; Cactinomycin; Calusterone; Caracemide; Carbetimer; Carboplatin (a platinum-containing regimen); Carmustine; Carubicin Hydrochloride; Carzelesin; Cedefingol; Ceritinib; Chlorambucil; Cirolemycin; Cisplatin (a platinum-containing regimen); Cladribine; Crisnatol Mesylate; Crizotinib; Cyclophosphamide; Cytarabine; Dacarbazine; Dactinomycin; Daunorubicin; Decitabine; Dexormaplatin; Dezaguanine; Diaziquone; Docetaxel (TAXOTERE); Doxorubicin; Droloxifene; Dromostanolone; Duazomycin; Edatrexate; Eflornithine; Elsamitrucin; Enloplatin; Enpromate; Epipropidine; Epirubicin; Erbulozole; Erlotinib (TARCEVA), Esorubicin; Estramustine; Etanidazole; Etoposide; Etoprine; Fadrozole; Fazarabine; Fenretinide; Floxuridine; Fludarabine; 5-Fluorouracil; Fluorocitabine; Fosquidone; Fostriecin; Gefitinib (IRESSA), Gemcitabine; Hydroxyurea; Idarubicin; Ifosfamide; Ilmofosine; Imatinib mesylate (GLEEVAC); Interferon alpha-2a; Interferon alpha-2b; Interferon alpha-n1; Interferon alpha-n3; Interferon beta-I a; Interferon gamma-I b; Iproplatin; Irinotecan; Lanreotide; Lenalidomide (REVLIMID, REVIMID); Letrozole; Leuprolide; Liarozole; Lometerxol; Lomustine; Losoxantrone; Masoprocol; Maytansine; Mechlorethamine; Megestrol; Melengestrol; Melphalan; Menogaril; Mercaptopurine; Methotrexate; Metoprine; Meturedepa; Mitindomide; Mitocarcin; Mitocromin; Mitogillin; Mitomalcin; Mitomycin; Mitosper; Mitotane; Mitoxantrone; Mycophenolic Acid; Nocodazole; Nogalamycin; Ormaplatin; Oxisuran; Paclitaxel; Pemeterxed (ALIMTA), Pegaspargase; Peliomycin; Pentamustine; Pentomone; Peplomycin; Perfosfamide; Pipobroman; Piposulfan; Piritrexim Isethionate; Piroxantrone; Plicamycin; Plomestane; Porfimer; Porfiromycin; Prednimustine; Procarbazine; Puromycin; Pyrazofurin; Riboprine; Rogletimide; Safingol; Semustine; Simtrazene; Sitogluside; Sparfosate; Sparsomycin; Spirogermanium; Spiromustine; Spiroplatin; Streptonigrin; Streptozocin; Sulofenur; Talisomycin; Tamsulosin; Taxol; Taxotere; Tecogalan; Tegafur; Teloxantrone; Temoporfin; Temozolomide (TEMODAR); Teniposide; Teroxirone; Testolactone; Thalidomide (THALOMID) and derivatives thereof; Thiamiprine; Thioguanine; Thiotepa; Tiazofurin; Tirapazamine; Topotecan; Toremifene; Trestolone; Triciribine; Trimeterxate; Triptorelin; Tubulozole; Uracil Mustard; Uredepa; Vapreotide; Verteporfin; Vinblastine; Vincristine; Vindesine; Vinepidine; Vinglycinate; Vinleurosine; Vinorelbine; Vinrosidine; Vinzolidine; Vorozole; Zeniplatin; Zinostatin; Zorubicin.

In some embodiments, one or more anti-cancer agents may be administered to a subject before, in combination with, or after administration of the antigens (e.g., an ALK variant), an ALK variant within a vesicle (e.g., ICMV) for the treatment of cancer (e.g., a solid tumor cancer). In certain embodiments, the anti-cancer agent Crizotinib may be administered to a subject before, in combination with, or after administration of the antigens (e.g., an ALK variant), an ALK variant within a vesicle (e.g., ICMV) for the treatment of cancer (e.g., a solid tumor cancer, non-small cell lung carcinoma (NSCLC), anaplastic large cell lymphoma, neuroblastoma). In certain embodiments, the anti-cancer agent Ceritinib may be administered to a subject before, in combination with, or after administration of the antigens (e.g., an ALK variant), an ALK variant within a vesicle (e.g., ICMV) for the treatment of cancer (e.g., a solid tumor cancer, non-small cell lung carcinoma (NSCLC), anaplastic large cell lymphoma, neuroblastoma). In certain embodiments, the anti-cancer agent Ceritinib may be administered to a subject before, in combination with, or after administration of the antigens (e.g., an ALK variant), an ALK variant within a vesicle (e.g., ICMV) for the treatment of anaplastic large cell lymphoma.

The anti-cancer agent may be an enzyme inhibitor including without limitation a tyrosine kinase inhibitor, a cyclin-dependent kinase (CDK) inhibitor, a mitogen-activated protein (MAP) kinase inhibitor, or an epidermal growth factor receptor (EGFR) inhibitor. The tyrosine kinase inhibitor may be without limitation Crizotinib, Ceritinib, Genistein (4′,5,7-trihydroxyisoflavone), Tyrphostin 25 (3,4,5-trihydroxyphenyl), methylene]-propanedinitrile, Herbimycin A, Daidzein (4′,7-dihydroxyisoflavone), AG-126, trans-1-(3′-carboxy-4′-hydroxyphenyl)-2-(2″,5″-dihydroxy-phenyl)ethane, or HDBA (2-Hydroxy-5-(2,5-Dihydroxybenzylamino)-2-hydroxybenzoic acid. The CDK inhibitor may be without limitation p21, p27, p57, p15, p16, p18, or p19. The MAP kinase inhibitor may be without limitation KY12420 (C.sub.23H.sub.24O.sub.8), CNI-1493, PD98059, or 4-(4-Fluorophenyl)-2-(4-methylsulfinyl phenyl)-5-(4-pyridyl) 1H-imidazole. The EGFR inhibitor may be without limitation erlotinib (TARCEVA), gefitinib (IRESSA), WH1-P97 (quinazoline derivative), LFM-A12 (leflunomide metabolite analog), ABX-EGF, lapatinib, canertinib, ZD-6474 (ZACTIMA), AEE788, and AG1458.

The anti-cancer agent may be a vascular endothelial growth factor (VEGF) inhibitor including without limitation bevacizumab (AVASTIN), ranibizumab (LUCENTIS), pegaptanib (MACUGEN), sorafenib, sunitinib (SUTENT), vatalanib, ZD-6474 (ZACTIMA), anecortave (RETAANE), squalamine lactate, and semaphorin.

The anti-cancer agent may be an antibody or an antibody fragment including without limitation an antibody or an antibody fragment including but not limited to bevacizumab (AVASTIN), trastuzumab (HERCEPTIN), alemtuzumab (CAMPATH, indicated for B cell chronic lymphocytic leukemia,), gemtuzumab (MYLOTARG, hP67.6, anti-CD33, indicated for leukemia such as acute myeloid leukemia), rituximab (RITUXAN), tositumomab (BEXXAR, anti-CD20, indicated for B cell malignancy), MDX-210 (bispecific antibody that binds simultaneously to HER-2/neu oncogene protein product and type I Fc receptors for immunoglobulin G (IgG) (Fc gamma RI)), oregovomab (OVAREX, indicated for ovarian cancer), edrecolomab (PANOREX), daclizumab (ZENAPAX), palivizumab (SYNAGIS, indicated for respiratory conditions such as RSV infection), ibritumomab tiuxetan (ZEVALIN, indicated for Non-Hodgkin's lymphoma), cetuximab (ERBITUX), MDX-447, MDX-22, MDX-220 (anti-TAG-72), IOR-05, IOR-T6 (anti-CD1), IOR EGF/R3, celogovab (ONCOSCINT OV103), epratuzumab (LYMPHOCIDE), pemtumomab (THERAGYN), and Gliomab-H (indicated for brain cancer, melanoma).

Immunomodulatory Agents

As used herein, an immunomodulatory agent is an agent that stimulates (i.e., an immunostimulatory agent) or inhibits (i.e., an immunoinhibitory agent) an immune response in a subject to whom it is administered, whether alone or in combination with another agent.

Immunostimulatory Agents

As used herein, an immunostimulatory agent is an agent that stimulates an immune response (including enhancing a pre-existing immune response) in a subject to whom it is administered, whether alone or in combination with another agent. Examples include antigens, adjuvants (e.g., TLR ligands such as imiquimod and resiquimod, imidazoquinolines, nucleic acids including an unmethylated CpG dinucleotide, monophosphoryl lipid A (MPLA) or other lipopolysaccharide derivatives, single-stranded or double-stranded RNA, flagellin, muramyl dipeptide), cytokines including interleukins (e.g., IL-2, IL-7, IL-15 (or superagonist/mutant forms of these cytokines), IL-12, IFN-gamma, IFN-alpha, GM-CSF, FLT3-ligand, etc.), AMD3100, immunostimulatory antibodies (e.g., anti-CD40, ipilimumab, anti-CTLA-4, anti-CD28, anti-CD3, or single chain/antibody fragments of these molecules), and PD-1 inhibitors. The term “PD-1 inhibitor” refers to any agent that inhibits the molecular pathway of PD-1. For example, a PD-1 inhibitor can be an antibody that binds to the PD-1 receptor to block ligand binding to PD-1 (e.g., an anti-PD-1 antibody, nivolumab, and pembrolizumab). A PD-1 inhibitor can also be an antibody that binds to PD-L1 or PD-L2, each of which is a ligand of PD-1, to prevent it from binding to the PD-1 receptor (e.g., an anti-PD-L1 antibody (e.g., BMS-936559 and MPDL3280A) and an anti-PD-L2 antibody (see, e.g., Patent Application Publication No.: WO 2010036959 (see, e.g., p. 79, ¶ [0253] through p. 101, ¶ [0296]))). Immunostimulatory agents as described herein specifically exclude CureTech's anti-PD-1 antibody CT-011 as described in Patent and Patent Application Publication Nos.: U.S. Pat. No. 8,686,119, WO 2013014668, and WO 2009101611.

Examples of anti-CD40 antibodies include antibodies described in the following Patent and Patent Application Publication Nos.: US 20030059427 (see, e.g., p. 15, ¶ [0157] through p. 20, ¶ [0212]), WO 2013034904 (see, e.g., p. 58, line 4 through p. 102, line 20), WO 2003029296 (see, e.g., p. 30, line 20 through p. 34, line 16), U.S. Pat. No. 8,637,032 (see, e.g., column 252, line 55 through column 254, line 37), WO 2002028905 (see, e.g., p. 20, line 18 through p. 32, line 30), U.S. Pat. No. 8,778,345 (see, e.g., column 48, line 31 through column 54, line 38), WO 1997031025 (see, e.g., p. 14, line 6 through p. 31, line 26), WO 2012125569 (see, e.g., p. 25, line 33 through p. 27, line 14), WO 2011123489 (see, e.g., p. 93, ¶ [00339] through p. 109, ¶ [00145]), CA 2544949 (see, e.g., p. 78, line 26 through p. 122, line 21), WO 2014070934 (see, e.g., p. 86, line 4 through p. 103, line 4), US 20140093497 (see, e.g., p. 12, ¶ [0112] through p. 13, ¶ [0118]), WO 2010104761 (see, e.g., p. 37, line 3 through p. 66, line 29), U.S. Pat. No. 8,591,900 (see, e.g., column 60, line 14 through column 80, line 29), WO 2007124299 (see, e.g., 68, line 29 through p. p. 88, line 17), U.S. Pat. No. 7,445,780 (see, e.g., column 22, line 29 through column 36, line 39), WO 2006073443 (see, e.g., p. 82, line 6 through p. 89, line 12), WO 2005044294 (see, e.g., p. 137, line 19 through p. 158, line 15), U.S. Pat. No. 5,677,165 (see, e.g., column 11, line 45 through column 18, line 6), WO 2001083755 (see, e.g., p. 39, line 4 through p. 47, line 2), US 20080057070 (see, e.g., p. 26, ¶ [0176] through p. 47, ¶ [0296]), U.S. Pat. No. 7,172,759 (see, e.g., column 9, line 5 through column 12, line 58), WO 2006128103 (see, e.g., p. 75, ¶ [00244] through p. 84, ¶ [000255]), WO 2001016180 (see, e.g., p. 79, line 21 through p. 89, line 14), WO 2003040170 (see, e.g., p. 76, ¶ [0248] through p. 141, ¶ [0239]), U.S. Pat. No. 6,312,693 (see, e.g., column 8, line 51 through column 34, line 45), U.S. Pat. No. 8,492,531 (see, e.g., column 47, line 46 through column 58, line 31), U.S. Pat. No. 8,551,485 (see, e.g., column 78, line 15 through column 85, line 7), U.S. Pat. No. 6,838,261 (see, e.g., column 26, line 10 through column 34, line 26), EP 2243492 (see, e.g., p. 26, ¶ [0144] through p. 37, ¶ [0219]), and EP 2011802 (see, e.g., p. 12, ¶ [0047] through p. 40, ¶ [0127]), each of which is incorporated herein by reference in its entirety.

Examples of anti-CTLA-4 antibodies include ipilimumab (see, e.g., Patent No.: U.S. Pat. No. 6,682,736 (see, e.g., column 34, line 40 through colum 48, line 6)) and antibodies described in the following Patent and Patent Application Publication Nos.: WO 2012120125 (see, e.g., p. 13, line 1 through p. 27, line 18), U.S. Pat. No. 8,017,114 (see, e.g., column 46, line 40 through column 74, line 12), WO 2001014424 (see, e.g., p. 65, line 21 through p. 96, line 15), and WO 2000037504 (see, e.g., p. 56, line 25 through p. 86, line 31), each of which is incorporated herein by reference in its entirety.

Immunoinhibitory Agents

As used herein, an immunoinhibitory agent is an agent that inhibits an immune response in a subject to whom it is administered, whether alone or in combination with another agent. Examples include immunoinhibitory antibodies (e.g., anti-CD3, or single chain/antibody fragments of this molecule), steroids, retinoic acid, dexamethasone, cyclophosphamide, and other immunosuppressants.

Other immunomodulatory agents include cell-surface makers and antibodies that target cell-surface makers. Examples of immunomodulatory agents such as cell-surface makers and antibodies that target cell-surface makers include anti-LAG-3/CD223 antibodies (such as C9B7W (UniProt ID No. P18627) and those described in the Patent and Patent Application Publication Nos.: WO 2010019570 (see, p. 73, line 4 through e.g., p. 97, line 10), WO 2014008218 (see, e.g., p. 57, lin 20 through p. 65, line 17), and WO 2008132601 (see, e.g., p. 15, line 13 through p. 28, line 17)), anti-VISTA/PD-L3 antibodies (such as those described in the Patent and Patent Application Publication Nos.: US 20140105912 (see, e.g., p. 71, ¶ [0601] through p. 87, ¶ [0755]), U.S. Pat. No. 8,236,304 (see, e.g., column 17, line 7 through column 18, line 48), and US 20110027278 (see, e.g., p. 39, ¶ [0302] through p. 43, ¶ [0333])), anti-B7-H5 antibodies (such as those described in the Patent Application Publication No.: US 20080248007 (see, e.g., p. 9, ¶ [0087] through p. 10, ¶ [0094])), anti-OX40 antibodies (such as those described in the Patent Application Publication No.: WO 2013130102 (see, e.g., p. 31, ¶ [0101] through p. 41, ¶ [0124])), anti-CD28 antibodies (such as those described in the Patent Application Publication No.: EP0440373 (see, e.g., p. 4, line 45 through p. 8, line 37)), anti-GITR antibodies (such as those described in the Patent and Patent Application Publication Nos.: WO 2007133822 (see, e.g., p. 48, line 16 through p. 52, line 18), WO 2009009116 (see, e.g., p. 52, line 30 through p. 56, line 6), WO 2004107618 (see, e.g., p. 78, ¶ [0199] through p. 105, ¶ [0261]), WO 2006105021 (see, e.g., p. 70, line 21 through p. 80, line 31), U.S. Pat. No. 7,812,135 (see, e.g., column 55, line 52 through column 66, line 38), and U.S. Pat. No. 8,591,886 (see, e.g., column 41, line 15 through column 44, line 20)), anti-4-1BB/CD137 antibodies (such as those described in the Patent No: U.S. Pat. No. 8,716,452 (see, e.g., column 13, line 55 through column 20, line 62)), 4-1BB ligands (such as those described in the Patent Application Publication Nos.: WO 1994026290 (see, e.g., 21, line 23 through p. 32, line 33), US 20060110802 (see, e.g., p. 9, ¶ [0098] through p. 16, ¶ [0167]), WO 1999036093 (see, e.g., p. 18, line 5 through p. 56, line 17), WO 2010132389 (see, e.g., p. 30, ¶ [00134] through p. 41, ¶ [00166]), WO 2012145183 (see, e.g., p. 43, line 26 through p. 64, line 12), US 20080008716 (see, e.g., p. 3, ¶ [0042] through p. 7, ¶ [0070]), WO 2004010947 (see, e.g., p. 13, line 12 through p. 23, line 19), and US 20070286860 (see, e.g., p. 27, ¶ [0172] through p. 31, ¶ [200])), anti-BTLA antibodies (such as those described in the Patent and Patent Application Publication Nos.: WO 2010106051 (see, e.g., p. 35, line through p. 35, line 8), WO 2008076560 (see, e.g., p. 85, line 2 through p. 97, line 11), U.S. Pat. No. 8,349,320 (see, e.g., column 47, line 62 through column 72, line 26), and U.S. Pat. No. 8,563,694 (see, e.g., column 56, line 25 through column 65, line 45)), anti-TIM-3/HAVCR2 antibodies (such as those described in the Patent and Patent Application Publication Nos.: U.S. Pat. No. 8,841,418 (see, e.g., column 36, line 45 through column 46, line 47), EP 2417984 (see, e.g., p. 19, ¶ [0137] through p. 28, ¶ [0206]), WO 2014022332 (see, e.g., p. 51, ¶ [00191] through p. 54, ¶ [00202]), and U.S. Pat. No. 8,697,069 (see, e.g., p. 40, line 26 through p. 50, line 37)), anti-KIR antibodies (such as those described in the Patent and Patent Application Publication Nos.: WO 2014066532 (see, e.g., p. 25, line 30 through p. 57, line 17), EP 2446897 (see, e.g., p. 45, ¶ [0294] through p. 47, ¶ [0309]), WO 2014055648 (see, e.g., pp. 24-49), and US 20140302052 (see, e.g., p. 2, ¶ [0021] through p. 4, ¶ [0044])), anti-Flt3/CD135 antibodies (such as those described in the Patent and Patent Application Publication Nos.: U.S. Pat. No. 6,291,661 (see, e.g., column 23, line 36 through column 38, line 23), EP 0754230 (see, e.g., p. 13, ¶ [0099] through p. 20, ¶ [0138]), EP 0992584 (see, e.g., p. 25, line 28 through p. 30, line 45), and WO 2011076922 (see, e.g., p. 95, ¶ [000269] through p. 82, ¶ [000233])), anti-FasL antibodies (such as those described in the Patent and Patent Application Publication Nos.: US 20100266577 (see, e.g., p. 7, ¶ [0089] through p. 10, ¶ [0122]), US 20070142456 (see, e.g., p. 6, ¶ [0054] through p. 8, ¶ [0066]), WO 2011066211 (see, e.g., pp. 38-97), US 20020187534 (see, e.g., p. 5, ¶ [0061] through p. 10, ¶ [0112]), WO 1999036079 (see, e.g., p. 53, line 15 through p. 60, line 19), and WO 1997033617 (see, e.g., p. 18, line 1 through p. 25, line 17)), and anti-CD25 antibodies (such as those described in the Patent and Patent Application Publication Nos.: WO 2006108670 (see, e.g., pp. 4-8), U.S. Pat. No. 8,182,812 (see, e.g., column 43, line 6 through column 54, line 15), and CA 2585776 (see, e.g., p. 31, ¶ [00100] through p. 52, ¶ [00163])).

Other immunomodulatory agents include cytokines or antibodies that target cytokine receptors. Examples of immunomodulatory agents such as cytokines and antibodies that target cytokine receptors include GM-CSF (such as those described in the Patent and Patent Application Publication Nos.: WO 2013074489 (see, e.g., p. 52, line 29 through p. 61, line 26), U.S. Pat. No. 5,891,429 (see, e.g., column 12, line 1 through column 28, line 21), and WO 1989010403 (see, e.g., p. 5, line 35 through p. 13, line 21)), anti-GM-CSF-receptor (R) antibodies (such as those described in the Patent and Patent Application Publication Nos.: WO 1994011404 (see, e.g., pp. 9-28), U.S. Pat. No. 8,263,075 (see, e.g., column 29, line 25 through column 49, line 26), and U.S. Pat. No. 5,932,704 (see, e.g., column 2, line 55 through column 12, line 67)), IL-2 (such as those described in the Patent and Patent Application Publication Nos.: WO 2013130102 (see, e.g., p. 31, ¶ [0101] through p. 41, ¶ [0124]), U.S. Pat. No. 8,349,311 (see, e.g., column 21, line 31 through column 31, line 9), WO 2005007121 (see, e.g., p. 30, line 29 through p. 43, line 31), and WO 1991002000 (see, e.g., pp. 2-7)), anti-IL-2-R antibodies (such as those described in the Patent Application Publication No.: WO 1989009622 (see, e.g., p. 18, line 4 through p. 33, line 12)), IL-7 (such as those described in the Patent and Patent Application Publication Nos.: WO 2012031115 (see, e.g., p. 77, ¶ [00240] through p. 102, ¶ [00347]), WO 2013074489 (see, e.g., p. 52, line 30 through p. 61, line 26), U.S. Pat. No. 7,323,549 (see, e.g., column 12, line 14 through column 24, line 37), and U.S. Pat. No. 8,338,575 (see, e.g., column 11, line 60 through column 24, line 18)), anti-IL-7-R antibodies, IL-21 (as those described in the Patent Application Publication No.: WO 2013169693 (see, e.g., p. 44, line 3 through p. 73, line 15)), anti-IL-21-R antibodies, IL-12 (as those described in the Patent No.: U.S. Pat. No. 8,765,462 (see, e.g., column 27, line 15 through column 50, line 16)), anti-IL-12-R antibodies, IL-15, anti-IL-15-R antibodies, IL-18, anti-IL-8-R antibodies, and anti-IDO antibodies.

Yet other immunomodulatory agents include kinase inhibitors such as crizotinib (a tyrosine kinase inhibitor; see, e.g., Patent Application Publication No.: WO 2013017989 (see, e.g., pp. 54-69)) and ceritinib (a tyrosine kinase inhibitor; see, e.g., Patent Application Publication Nos.: WO 2012082972 (see, e.g., p. 11, line 6 through p. 14, line 17) and WO 2008073687 (see, e.g., p. 34, ¶ [0089] through p.144, ¶ [0151])), COX-2 inhibitors such as celecoxib (such as those described in the Patent and Patent Application Publication Nos.: WO 2000032189 (see, e.g., p. 38, line 17 through p. 61, line 20), U.S. Pat. No. 6,127,545 (see, e.g., column 7, line 30 through column 17, line 67), WO 2002028270 (see, e.g., p. 37, line 27 through p. 45, line 28), U.S. Pat. No. 6,403,630 (see, e.g., column 16, line 29 through column 17, line 7), and U.S. Pat. No. 5,972,986 (see, e.g., column 5, line 9 through column 16, line 44)), SOCS-1 inhibitors (e.g., PI3K or Jak inhibitors), heat shock proteins (HSP) (such as those described in the Patent and Patent Application Publication Nos.: U.S. Pat. No. 7,678,803 (see, e.g., column 79, line 20 through column 158, line 55), U.S. Pat. No. 7,608,635 (see, e.g., column 93, line 55 through column 108, line 40), U.S. Pat. No. 8,318,790 (see, e.g., column 158, line 51 through column 196, line 20), and US 20130184336 (see, e.g., p. 5, ¶ [0075] through p. 6, ¶ [0093])), HSP inhibitors (such as those described in the Patent No.: U.S. Pat. No. 7,776,849 (see, e.g., column 32, line 47 through column 50, line 67)), and anti-galectin-1 antibodies (such as those described in the Patent Application Publication Nos.: WO 2012131079 (see, e.g., pp. 24-35), WO 2014070214 (see, e.g., p. 42, line 15 through p. 53, line 13), and WO 2014043708 (see, e.g., p. 27, ¶ [00140] through p. 36, ¶ [00199])).

The disclosures of the aforementioned Patent and Patent Application Publication Nos. are incorporated herein by reference in their entireties.

Cancer Antigens

A cancer antigen is an antigen that is expressed preferentially by cancer cells (i.e., it is expressed at higher levels in cancer cells than on non-cancer cells) and in some instances it is expressed solely by cancer cells. The cancer antigen may be expressed within a cancer cell or on the surface of the cancer cell. The cancer antigen may be MART-1/Melan-A, gp100, adenosine deaminase-binding protein (ADAbp), FAP, cyclophilin b, colorectal associated antigen (CRC)-0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T-cell receptor/CD3-zeta chain, telomerase, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05). The cancer antigen may be selected from the group consisting of GAGE-1, GAGE-2, GAGE-3, GAGE-4, GAGE-5, GAGE-6, GAGE-7, GAGE-8, GAGE-9. The cancer antigen may be selected from the group consisting of BAGE, RAGE, LAGE-1, NAG, GnT-V, MUM-1, CDK4, tyrosinase, p53, MUC family, HER2/neu, p21 ras, RCAS1, .alpha.-fetoprotein, E-cadherin, .alpha.-catenin, .beta.-catenin, .gamma.-catenin, p120ctn, gp100.sup.Pme1117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus (HPV) proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, and c-erbB-2.

Anti-Infective Agents

The potentiating agent may be an anti-infective agent including without limitation an anti-bacterial agent (e.g., an anti-mycobacterial agent), an anti-viral agent, an anti-parasitic agent, and an anti-fungal agent.

Anti-bacterial agents may be without limitation beta-lactam antibiotics, penicillins (such as natural penicillins, aminopenicillins, penicillinase-resistant penicillins, carboxy penicillins, ureido penicillins), cephalosporins (first generation, second generation, and third generation cephalosporins), other beta-lactams (such as imipenem, monobactams), beta-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, or quinolines.

Other anti-bacterials may be without limitation Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfas alazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; or Zorbamycin. Anti-mycobacterial agents may be without limitation Myambutol (Ethambutol Hydrochloride), Dapsone (4,4′-diaminodiphenylsulfone), Paser Granules (aminosalicylic acid granules), Priftin (rifapentine), Pyrazinamide, Isoniazid, Rifadin (Rifampin), Rifadin IV, Rifamate (Rifampin and Isoniazid), Rifater (Rifampin, Isoniazid, and Pyrazinamide), Streptomycin Sulfate or Trecator-SC (Ethionamide).

Anti-Viral Agents

Anti-viral agents may be without limitation amantidine and rimantadine, ribivarin, acyclovir, vidarabine, trifluorothymidine, ganciclovir, zidovudine, retinovir, and interferons. Anti-viral agents may be without limitation further include Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime or integrase inhibitors.

Anti-Fungal Agents

Anti-fungal agents may be without limitation imidazoles and triazoles, polyene macrolide antibiotics, griseofulvin, amphotericin B, and flucytosine. Antiparasite agents include heavy metals, antimalarial quinolines, folate antagonists, nitroimidazoles, benzimidazoles, avermectins, praxiquantel, ornithine decarboxylase inhibitors, phenols (e.g., bithionol, niclosamide); synthetic alkaloid (e.g., dehydroemetine); piperazines (e.g., diethylcarbamazine); acetanilide (e.g., diloxanide furonate); halogenated quinolines (e.g., iodoquinol (diiodohydroxyquin)); nitrofurans (e.g., nifurtimox); diamidines (e.g., pentamidine); tetrahydropyrimidine (e.g., pyrantel pamoate); or sulfated naphthylamine (e.g., suramin). Other anti-infective agents may be without limitation Difloxacin Hydrochloride; Lauryl Isoquinolinium Bromide; Moxalactam Disodium; Ornidazole; Pentisomicin; Sarafloxacin Hydrochloride; Protease inhibitors of HIV and other retroviruses; Integrase Inhibitors of HIV and other retroviruses; Cefaclor (CECLOR); Acyclovir (ZOVIRAX); Norfloxacin (NOROXIN); Cefoxitin (MEFOXIN); Cefuroxime axetil (CEFTIN); Ciprofloxacin (CIPRO); Aminacrine Hydrochloride; Benzethonium Chloride: Bithionolate Sodium; Bromchlorenone; Carbamide Peroxide; Cetalkonium Chloride; Cetylpyridinium Chloride: Chlorhexidine Hydrochloride; Clioquinol; Domiphen Bromide; Fenticlor; Fludazonium Chloride; Fuchsin, Basic; Furazolidone; Gentian Violet; Halquinols; Hexachlorophene: Hydrogen Peroxide; Ichthammol; Imidecyl Iodine; Iodine; Isopropyl Alcohol; Mafenide Acetate; Meralein Sodium; Mercufenol Chloride; Mercury, Ammoniated; Methylbenzethonium Chloride; Nitrofurazone; Nitromersol; Octenidine Hydrochloride; Oxychlorosene; Oxychlorosene Sodium; Parachlorophenol, Camphorated; Potassium Permanganate; Povidone-Iodine; Sepazonium Chloride; Silver Nitrate; Sulfadiazine, Silver; Symclosene; Thimerfonate Sodium; Thimerosal; or Troclosene Potassium.

### Subjects

The invention can be practiced in virtually any subject type that is likely to benefit from delivery of ALK variants as contemplated herein. In some embodiments, the subject is human. Subjects also include animals such as household pets (e.g., dogs, cats, rabbits, ferrets, etc.), livestock or farm animals (e.g., cows, pigs, sheep, chickens and other poultry), horses such as thoroughbred horses, and laboratory animals (e.g., mice, rats, rabbits). Subjects also include fish and other aquatic species.

The subjects to whom the ALK variants are delivered may be normal subjects. Alternatively they may have or may be at risk of developing a condition that can benefit from delivery of one or more particular ALK variants.

Such conditions include cancer (e.g., solid tumor cancers or non-solid cancer such as leukemias), infections (including infections localized to particular regions or tissues in the body), autoimmune disorders, allergies or allergic conditions, asthma, and transplant rejection.

Tests for diagnosing the conditions embraced by the invention are known in the art and will be familiar to the ordinary medical practitioner. These laboratory tests include without limitation, endoscopy, direct visualization, microscopic analyses, cultivation dependent tests (such as cultures), and nucleic acid detection tests. These include wet mounts, stain-enhanced microscopy, immune microscopy (e.g., FISH), hybridization microscopy, particle agglutination, enzyme-linked immunosorbent assays, urine screening tests, DNA probe hybridization, and serologic tests. The medical practitioner will generally also take a full history and conduct a complete physical examination in addition to running the laboratory tests listed above.

A subject having a cancer is a subject that has detectable cancer cells. A subject at risk of developing a cancer is a subject that has a higher than normal probability of developing cancer. These subjects include, for instance, subjects having a genetic abnormality that has been demonstrated to be associated with a higher likelihood of developing a cancer, subjects having a familial disposition to cancer, subjects exposed to cancer causing agents (i.e., carcinogens) such as tobacco, asbestos, or other chemical toxins, and subjects previously treated for cancer and in apparent remission. Cancers that a subject may have include solid tumor cancers and ALK⁺ cancers. Exemplary solid tumor cancers and ALK⁺ cancers include anaplastic large cell lymphoma, non-small-cell lung cancer, neuroblastoma, rhabdomyosarcoma, neuroectodermal cancer, glioblastoma, breast carcinoma, melanoma, inflammatory myofibroblastic tumor, soft tissue tumor, ALK expressing lymphoma, and ALK expressing lung, colon, or prostate carcinoma.

Other cancers that a subject may have include, but are not limited to, bladder cancer, pancreatic cancer, lung cancer, liver cancer, ovarian cancer, colon cancer, stomach cancer, breast cancer, prostate cancer, renal cancer, testicular cancer, thyroid cancer, uterine cancer, rectal cancer, a cancer of the respiratory system, a cancer of the urinary system, oral cavity cancer, skin cancer, leukemia, sarcoma, carcinoma, basal cell carcinoma, non-Hodgkin's lymphoma, acute myeloid leukemia (AML), chronic lymphocytic leukemia (CLL), B-cells chronic lymphocytic leukemia (B-CLL), multiple myeloma (MM), erythroleukemia, renal cell carcinoma, astrocytoma, oligoastrocytoma, biliary tract cancer, choriocarcinoma, CNS cancer, larynx cancer, small cell lung cancer, adenocarcinoma, giant (or oat) cell carcinoma, and squamous cell carcinoma.

Subjects having an infection are those that exhibit symptoms thereof including without limitation fever, chills, myalgia, photophobia, pharyngitis, acute lymphadenopathy, splenomegaly, gastrointestinal upset, leukocytosis or leukopenia, and/or those in whom infectious pathogens or byproducts thereof can be detected.

A subject at risk of developing an infection is one that is at risk of exposure to an infectious pathogen. Such subjects include those that live in an area where such pathogens are known to exist and where such infections are common. These subjects also include those that engage in high risk activities such as sharing of needles, engaging in unprotected sexual activity, routine contact with infected samples of subjects (e.g., medical practitioners), people who have undergone surgery, including but not limited to abdominal surgery.

### Pharmaceutical Compositions and Preparations

In some embodiments, pharmaceutical compositions of the invention contain a vesicle (e.g., an ICMV) including to an ALK variant. In addition to vesicles (e.g., ICMV) including an ALK variant, the pharmaceutical compositions may contain one or more adjuvants and/or potentiating agents. Examples of adjuvants and potentiating agents are described herein. The pharmaceutical compositions may also include pharmaceutically acceptable carrier or excipient, which can be formulated by methods known to those skilled in the art.

In some embodiments, a pharmaceutical composition of the invention may contain a vesicle (e.g., an ICMV) including an ALK variant. In some embodiments, a pharmaceutical composition of the invention containing ALK-ICMV may further contain an adjuvant, e.g., monophosphoryl lipid A (MPLA)) and/or a potentiating agent (e.g., an antibody). In other desirable embodiments, the vesicles (e.g., ICMV) including an ALK variant are administered in the absence of an adjuvant.

Effective Amounts, Regimens, Formulations

The ALK variants are administered in the form of stabilized MLVs or ICMVs including one or more conjugated ALK variants, and in effective amounts. An effective amount is a dosage of the ALK variant sufficient to provide a medically desirable result. The effective amount will vary with the desired outcome, the particular condition being treated or prevented, the age and physical condition of the subject being treated, the severity of the condition, the duration of the treatment, the nature of the concurrent or combination therapy (if any), the specific route of administration and like factors within the knowledge and expertise of the health practitioner. It is preferred generally that a maximum dose be used, that is, the highest safe dose according to sound medical judgment.

For example, if the subject has a tumor, an effective amount may be that amount that reduces the tumor volume or load (as for example determined by imaging the tumor). Effective amounts may also be assessed by the presence and/or frequency of cancer cells in the blood or other body fluid or tissue (e.g., a biopsy). If the tumor is impacting the normal functioning of a tissue or organ, then the effective amount may be assessed by measuring the normal functioning of the tissue or organ.

In some instances the effective amount is the amount required to lessen or eliminate one or more, and preferably all, symptoms. For example, in a subject having an infection, an effective amount of an ALK variant may be that amount that lessens or eliminate the symptoms associated with the infection. These may include fever and malaise. It will be understood that in some instances the invention contemplates single administration of an ALK variant and in some instances the invention contemplates multiple administrations of an ALK variant. As an example, an antigen may be administered in a prime dose and a boost dose, although in some instances the invention provides sufficient delivery of the antigen, and optionally an adjuvant, that no boost dose is required.

The invention provides pharmaceutical compositions. Pharmaceutical compositions are sterile compositions that include the vesicles including an ALK variant preferably in a pharmaceutically-acceptable carrier. The term “pharmaceutically-acceptable carrier” means one or more compatible solid or liquid filler, diluents or encapsulating substances which are suitable for administration to a human or other subject contemplated by the invention.

The term “carrier” denotes an organic or inorganic ingredient, natural or synthetic, with which vesicles including an ALK variant are combined to facilitate administration. The components of the pharmaceutical compositions are comingled in a manner that precludes interaction that would substantially impair their desired pharmaceutical efficiency.

Suitable buffering agents include acetic acid and a salt (1-2% W/V); citric acid and a salt (1-3% W/V); boric acid and a salt (0.5-2.5% W/V); and phosphoric acid and a salt (0.8-2% W/V). Suitable preservatives include benzalkonium chloride (0.003-0.03% W/V); chlorobutanol (0.3-0.9% W/V); and parabens (0.01-0.25% W/V).

Unless otherwise stated herein, a variety of administration routes are available. The particular mode selected will depend, of course, upon the particular condition being treated and the dosage required for therapeutic efficacy. The methods provided, generally speaking, may be practiced using any mode of administration that is medically acceptable, meaning any mode that produces effective levels of a desired response without causing clinically unacceptable adverse effects. One mode of administration is a parenteral route. The term “parenteral” includes subcutaneous injections, intravenous, intramuscular, intraperitoneal, intra sternal injection or infusion techniques. Other modes of administration include oral, mucosal, rectal, vaginal, sublingual, intranasal, intratracheal, inhalation, ocular, and transdermal. For oral administration, the compounds can be formulated readily by combining the vesicles with pharmaceutically acceptable carriers well known in the art. Such carriers enable formulation as tablets, pills, dragees, capsules, liquids, gels, syrups, slurries, films, suspensions and the like, for oral ingestion by a subject to be treated. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). Optionally the oral formulations may also be formulated in saline or buffers for neutralizing internal acid conditions or may be administered without any carriers.

Pharmaceutical preparations which can be used orally include push-fit capsules made of gelatin, as well as soft, sealed capsules made of gelatin and a plasticizer, such as glycerol or sorbitol. The push-fit capsules can contain the vesicles suspended in suitable liquids, such as aqueous solutions, buffered solutions, fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compositions may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

When it is desirable to deliver the compositions of the invention systemically, they may be formulated for parenteral administration by injection, e.g., by bolus injection or continuous infusion. Formulations for injection may be presented in unit dosage form, e.g., in ampoules or in multi-dose containers. Pharmaceutical parenteral formulations include aqueous solutions of the ingredients. Aqueous injection suspensions may contain substances which increase the viscosity of the suspension, such as sodium carboxymethyl cellulose, sorbitol, or dextran. Alternatively, suspensions of vesicles may be prepared as oil-based suspensions. Suitable lipophilic solvents or vehicles include fatty oils such as sesame oil, or synthetic fatty acid esters, such as ethyl oleate or triglycerides.

Alternatively, the vesicles may be in powder form or lyophilized form for constitution with a suitable vehicle, e.g., sterile pyrogen-free water, before use.

The compositions may also be formulated in rectal or vaginal compositions such as suppositories or retention enemas, e.g., containing conventional suppository bases such as cocoa butter or other glycerides.

Acceptable carriers and excipients in the pharmaceutical compositions do not reduce or prevent the uptake of vesicles (e.g., ICMV (e.g., ALK-ICMV)) and are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol.

The pharmaceutical compositions of the invention may be administered parenterally in the form of an injectable formulation. The pharmaceutical compositions for injection may be formulated using a sterile solution or any pharmaceutically acceptable liquid as a vehicle. The pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium).

The pharmaceutical compositions of the invention may be prepared in microcapsules, such as hydroxylmethylcellulose or gelatin-microcapsule and poly-(methylmethacrylate) microcapsule. The pharmaceutical compositions of the invention may also be prepared in other drug delivery systems such as liposomes, albumin microspheres, microemulsions, nano-particles, and nanocapsules. Such techniques are described in Remington: The Science and Practice of Pharmacy 22^{th} edition (2012). The pharmaceutical compositions to be used for in vivo administration must be sterile. This is readily accomplished by filtration through sterile filtration membranes.

Methods of Treatment Using a Vesicle (e.g., ICMV) including an ALK variant

The invention provides pharmaceutical compositions containing a vesicle (e.g., an ICMV) including an ALK variant that may be used to treat patients who are suffering from diseases and disorders, such as cancers and infections. In some embodiments, pharmaceutical compositions containing a vesicle (e.g., an ICMV) including an ALK variant may also contain one or more adjuvants and/or potentiating agents. In other embodiments, the composition is formulated for administration without an adjuvant. Examples of adjuvants and potentiating agents are described herein. The specific adjuvant and/or potentiating agent used may depend on the specific disease or disorder being treated. For example, a pharmaceutical composition containing a vesicle (e.g., an ICMV) including an ALK variant used to treat cancer (e.g., a solid tumor cancer or cervical cancer) may contain one or more anticancer agents as a potentiating agent.

A pharmaceutical composition of the invention containing a vesicle (e.g., an ICMV) including an ALK variant may be used to treat cancer, such as tumor cancers and ALK⁺ cancers. Exemplary solid tumor cancers and ALK⁺ cancers include anaplastic large cell lymphoma, non-small-cell lung cancer, neuroblastoma, rhabdomyosarcoma, neuroectodermal cancer, glioblastoma, breast carcinoma, melanoma, inflammatory myofibroblastic tumor, soft tissue tumor, ALK expressing lymphoma, and ALK expressing lung, colon, or prostate carcinoma.

Other cancers that may be treated with a pharmaceutical composition of the invention include, but are not limited to, bladder cancer, pancreatic cancer, lung cancer, liver cancer, ovarian cancer, colon cancer, stomach cancer, breast cancer, prostate cancer, renal cancer, testicular cancer, thyroid cancer, uterine cancer, rectal cancer, a cancer of the respiratory system, a cancer of the urinary system, oral cavity cancer, skin cancer, leukemia, sarcoma, carcinoma, basal cell carcinoma, non-Hodgkin's lymphoma, acute myeloid leukemia (AML), chronic lymphocytic leukemia (CLL), B-cells chronic lymphocytic leukemia (B-CLL), multiple myeloma (MM), erythroleukemia, renal cell carcinoma, astrocytoma, oligoastrocytoma, biliary tract cancer, choriocarcinoma, CNS cancer, larynx cancer, small cell lung cancer, adenocarcinoma, giant (or oat) cell carcinoma, and squamous cell carcinoma.

A pharmaceutical composition of the invention containing a vesicle (e.g., an ICMV) including an ALK variant may be used to treat infection, such as a bacterial infection, a viral infection, a fungal infection, a parasitic infection, and a mycobacterial infection.

For treating one or more of these diseases and disorders, the pharmaceutical composition may include a therapeutically effective amount of the vesicle (e.g., ICMV) including an ALK variant that is administered to patients in need thereof. As used herein, the term “therapeutically effective amount” refers to an amount effective to achieve the desired therapeutic effect. In particular, the therapeutic effective amount avoids adverse side effects.

### EXAMPLES

### Example 1 Amino Acid Sequences of Exemplary ALK Variants

| ALK variant 1: cytoplasmic domain of ALK having K93R substitution | |
|---|---|
| (SEQ ID NO: 3) | |
| 1 | VYRRKHQELQ AMQMELQSPE YKLSKLRTST IMTDYNPNYC FAGKTSSISD LKEVPRKNNT |
| 61 | LIRGLGHGAF GEVYEGQVSG MPNDPSPLQV AVRTLPEVCS EQDELDFLME ALIISKFNHQ |
| 121 | NIVRCIGVSL QSLPRFILLE LMAGGDLKSF LRETRPRPSQ PSSLAMLDLL HVARDIACGC |
| 181 | QYLEENHFIH RDIAARNCLL TCPGPGRVAK IGDFGMARDI YRASYYRKGG CAMLPVKWMP |
| 241 | PEAFMEGIFT SKTDTWSFGV LLWEIFSLGY MPYPSKSNQE VLEFVTSGGR MDPPKNCPGP |
| 301 | VYRIMTQCWQ HQPEDRPNFA IILERIEYCT QDPDVINTAL PIEYGPLVEE EEKVPVRPKD |
| 361 | PEGVPPLLVS QQAKREEERS PAAPPPLPTT SSGKAAKKPT AAEVSVRVPR GPAVEGGHVN |
| 421 | MAFSQSNPPS ELHRVHGSRN KPTSLWNPTY GSWFTEKPTK KNNPIAKKEP HERGNLGLEG |
| 481 | SCTVPPNVAT GRLPGASLLL EPSSLTANMK EVPLFRLRHF PCGNVNYGYQ QQGLPLEAAT |
| 541 | APGAGHYEDT ILKSKNSMNQ PGP |
| ALK variant 2 (SEQ ID NO: 4; amino acids 40-341 of SEQ ID NO: 3) | |
| 1 | CFAGKTSSIS DLKEVPRKNN TLIRGLGHGA FGEVYEGQVS GMPNDPSPLQ VAVRTLPEVC |
| 61 | SEQDELDFLM EALIISKFNH QNIVRCIGVS LQSLPRFILL ELMAGGDLKS FLRETRPRPS |
| 121 | QPSSLAMLDL LHVARDIACG CQYLEENHFI HRDIAARNCL LTCPGPGRVA KIGDFGMARD |
| 181 | IYRASYYRKG GCAMLPVKWM PPEAFMEGIF TSKTDTWSFG VLLWEIFSLG YMPYPSKSNQ |
| 241 | EVLEFVTSGG RMDPPKNCPG PVYRIMTQCW QHQPEDRPNF AIILERIEYC TQDPDVINTA |
| 301 | LP |
| ALK variant 3 (SEQ ID NO: 5; amino acids 40-409 of SEQ ID NO: 3) | |
| 1 | CFAGKTSSIS DLKEVPRKNN TLIRGLGHGA FGEVYEGQVS GMPNDPSPLQ VAVRTLPEVC |
| 61 | SEQDELDFLM EALIISKFNH QNIVRCIGVS LQSLPRFILL ELMAGGDLKS FLRETRPRPS |
| 121 | QPSSLAMLDL LHVARDIACG CQYLEENHFI HRDIAARNCL LTCPGPGRVA KIGDFGMARD |
| 181 | IYRASYYRKG GCAMLPVKWM PPEAFMEGIF TSKTDTWSFG VLLWEIFSLG YMPYPSKSNQ |
| 241 | EVLEFVTSGG RMDPPKNCPG PVYRIMTQCW QHQPEDRPNF AIILERIEYC TQDPDVINTA |
| 301 | LPIEYGPLVE EEEKVPVRPK DPEGVPPLLV SQQAKREEER SPAAPPPLPT TSSGKAAKKP |
| 361 | TAAEVSVRVP |
| ALK variant 4 (SEQ ID NO: 6; NPM protein (bold) attached to | |
| the N-terminus of ALK variant 1) | |
| 1 | MEDSMDMDMS PLRPQNYLFG CELKADKDYH FKVDNDENEH QLSLRTVSLG AGAKDELHIV |
| 61 | EAEAMNYEGS PIKVTLATLK MSVQPTVSLG GFEITPPVVL RLKCGSGPVH ISGQHLVVYR |
| 121 | RKHQELQAMQ MELQSPEYKL SKLRTSTIMT DYNPNYCFAG KTSSISDLKE VPRKNNTLIR |
| 181 | GLGHGAFGEV YEGQVSGMPN DPSPLQVAVR TLPEVCSEQD ELDFLMEALI ISKFNHQNIV |
| 241 | RCIGVSLQSL PRFILLELMA GGDLKSFLRE TRPRPSQPSS LAMLDLLHVA RDIACGCQYL |
| 301 | EENHFIHRDI AARNCLLTCP GPGRVAKIGD FGMARDIYRA SYYRKGGCAM LPVKWMPPEA |
| 361 | FMEGIFTSKT DTWSFGVLLW EIFSLGYMPY PSKSNQEVLE FVTSGGRMDP PKNCPGPVYR |
| 421 | IMTQCWQHQP EDRPNFAIIL ERIEYCTQDP DVINTALPIE YGPLVEEEEK VPVRPKDPEG |
| 481 | VPPLLVSQQA KREEERSPAA PPPLPTTSSG KAAKKPTAAE VSVRVPRGPA VEGGHVNMAF |
| 541 | SQSNPPSELH RVHGSRNKPT SLWNPTYGSW FTEKPTKKNN PIAKKEPHER GNLGLEGSCT |
| 601 | VPPNVATGRL PGASLLLEPS SLTANMKEVP LFRLRHFPCG NVNYGYQQQG LPLEAATAPG |
| 661 | AGHYEDTILK SKNSMNQPGP |
| ALK variant 5 (SEQ ID NO: 7; NPM protein (bold) attached to | |
| the N-terminus of ALK variant 2) | |
| 1 | MEDSMDMDMS PLRPQNYLFG CELKADKDYH FKVDNDENEH QLSLRTVSLG AGAKDELHIV |
| 61 | EAEAMNYEGS PIKVTLATLK MSVQPTVSLG GFEITPPVVL RLKCGSGPVH ISGQHLVCFA |
| 121 | GKTSSISDLK EVPRKNNTLI RGLGHGAFGE VYEGQVSGMP NDPSPLQVAV RTLPEVCSEQ |
| 181 | DELDFLMEAL IISKFNHQNI VRCIGVSLQS LPRFILLELM AGGDLKSFLR ETRPRPSQPS |
| 241 | SLAMLDLLHV ARDIACGCQY LEENHFIHRD IAARNCLLTC PGPGRVAKIG DFGMARDIYR |
| 301 | ASYYRKGGCA MLPVKWMPPE AFMEGIFTSK TDTWSFGVLL WEIFSLGYMP YPSKSNQEVL |
| 361 | EFVTSGGRMD PPKNCPGPVY RIMTQCWQHQ PEDRPNFAII LERIEYCTQD PDVINTALP |
| ALK variant 6 (SEQ ID NO: 8; NPM protein (bold) attached to | |
| the N-terminus of ALK variant 3) | |
| 1 | MEDSMDMDMS PLRPQNYLFG CELKADKDYH FKVDNDENEH QLSLRTVSLG AGAKDELHIV |
| 61 | EAEAMNYEGS PIKVTLATLK MSVQPTVSLG GFEITPPVVL RLKCGSGPVH ISGQHLVCFA |
| 121 | GKTSSISDLK EVPRKNNTLI RGLGHGAFGE VYEGQVSGMP NDPSPLQVAV RTLPEVCSEQ |
| 181 | DELDFLMEAL IISKFNHQNI VRCIGVSLQS LPRFILLELM AGGDLKSFLR ETRPRPSQPS |
| 241 | SLAMLDLLHV ARDIACGCQY LEENHFIHRD IAARNCLLTC PGPGRVAKIG DFGMARDIYR |
| 301 | ASYYRKGGCA MLPVKWMPPE AFMEGIFTSK TDTWSFGVLL WEIFSLGYMP YPSKSNQEVL |
| 361 | EFVTSGGRMD PPKNCPGPVY RIMTQCWQHQ PEDRPNFAII LERIEYCTQD PDVINTALPI |
| 421 | EYGPLVEEEE KVPVRPKDPE GVPPLLVSQQ AKREEERSPA APPPLPTTSS GKAAKKPTAA |
| 481 | EVSVRVP |
| ALK variant 7 (SEQ ID NO: 9) | |
| 1 | QELQAMQMEL QSPEYKLSKL RTSTIMTDYN PNYCFAGKTS SISDLKEVPR KNITLIRGLG |
| 61 | HGAFGEVYEG QVSGMPNDPS PLQVAVRTLP EVCSEQDELD FLMEALIISK FNHQNIVRCI |
| 121 | GVSLQSLPRF ILLELMAGGD LKSFLRETRP RPSQPSSLAM LDLLHVARDI ACGCQYLEEN |
| 181 | HFIHRDIAAR NCLLTCPGPG RVAKIGDFGM ARDIYRASYY RKGGCAMLPV KWMPPEAFME |
| 241 | GIFTSKTDTW SFGVLLWEIF SLGYMPYPSK SNQEVLEFVT SGGRMDPPKN CPGPVYRIMT |
| 301 | QCWQHQPEDR PNFAIILERI EYCTQDPDVI NTALPIEYGP LVEEEEK |

### Example 2 Overall T-Cell Response Stimulated by an ALK Variant Encapsulated in ICMVs

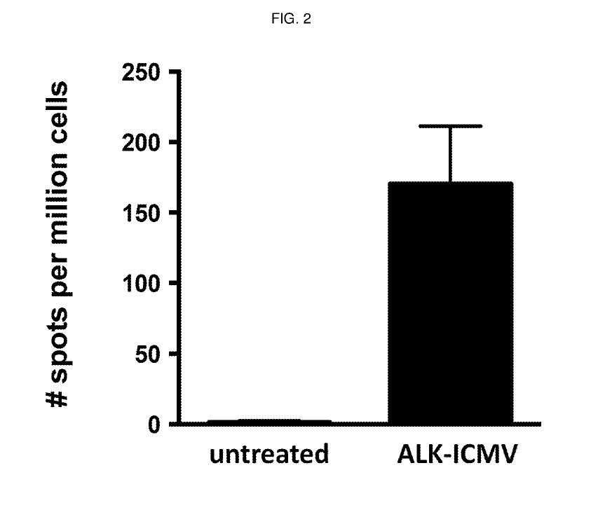

Mice were immunized with an ALK variant having the sequence of SEQ ID NO: 9 encapsulated in ICMVs. Blood was collected 7 days after immunization and assayed by Enzyme-Linked ImmunoSpot (ELISPOT). Cells were re-stimulated with a CD8 reactive peptide. ELISPOT results in FIG. 2 show that T-cell responses were found in cells collected from ALK-ICMV immunized mice, but not in untreated mice. This shows that ALK-ICMV can stimulate ALK-specific T-cell responses.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents and patent applications are herein incorporated by reference in their entirety to the same extent as if each individual publication, patent or patent application was specifically and individually indicated to be incorporated by reference in its entirety.

Other embodiments are within the following claims.

What is claimed is:

1. A composition comprising:
(a) a multilamellar lipid vesicle having crosslinks between lipid bilayers; and
(b) an anaplastic lymphoma kinase (ALK) variant.

2. The composition of claim 1, wherein said ALK variant is conjugated to a lipid.

3. The composition of claim 1 or 2, wherein said composition further comprises a nucleophosmin (NPM) protein or a fragment thereof.

4. The composition of claim 3, wherein said fragment of said NPM protein is an extracellular domain of said NPM protein.

5. The composition of claim 3 or 4, wherein said NPM protein is fused to said ALK variant.

6. The composition of claim 1 or 2, wherein said composition further comprises a tropomyosin (TMP3) protein or a fragment thereof.

7. The composition of claim 6, wherein said fragment of said TMP3 protein is an extracellular domain of said TMP3 protein.

8. The composition of claim 6 or 7, wherein said TMP3 protein is fused to said ALK variant.

9. The composition of claim 1 or 2, wherein said composition further comprises a 5-Aminoimidazole-4-carboxamide ribonucleotide formyltransferase/IMP cyclohydrolase (ATIC) protein or a fragment thereof.

10. The composition of claim 9, wherein said fragment of said ATIC protein is an extracellular domain of said ATIC protein.

11. The composition of claim 9 or 10, wherein said ATIC protein is fused to said ALK variant.

12. The composition of claim 1 or 2, wherein said composition further comprises a transforming growth factor (TGF) protein or a fragment thereof.

13. The composition of claim 12, wherein said fragment of said TGF protein is an extracellular domain of said TGF protein.

14. The composition of claim 12 or 13, wherein said TGF protein is fused to said ALK variant.

15. The composition of claim 1 or 2, wherein said composition further comprises an echinoderm microtubule-associated protein-like 4 (EML4) protein or a fragment thereof.

16. The composition of claim 15, wherein said fragment of said EML4 protein is an extracellular domain of said EML4 protein.

17. The composition of claim 15 or 16, wherein said EML4 protein is fused to said ALK variant.

18. The composition of any one of claims 1-17, wherein at least two lipid bilayers in the multilamellar lipid vesicle are covalently crosslinked to each other through headgroups that react with covalent crosslinkers to form the covalent crosslinks between lipid bilayers.

19. The composition of any one of claims 1-18, wherein said lipid bilayers comprise anionic and/or neutral lipids.

20. The composition of any one of claims 1-18, wherein said lipid bilayers comprise cationic lipids.

21. The composition of any one of claims 1-20, further comprising an adjuvant.

22. The composition of any one of claims 1-21, further comprising a potentiating agent.

23. The composition of claim 22, wherein said potentiating agent is a small molecule.

24. The composition of claim 23, wherein said small molecule is an anti-cancer agent.

25. The composition of claim 24, wherein said anti-cancer agent is Crizotinib.

26. The composition of claim 24, wherein said anti-cancer agent is Ceritinib.

27. The composition of claim 22, wherein said potentiating agent is an antibody.

28. The composition of claim 27, wherein said antibody is an immunomodulatory agent.

29. The composition of claim 28, wherein said immunomodulatory agent is selected from the group consisting of an anti-PD-1 antibody, an anti-PD-L1 antibody, and an anti-CTLA-4 antibody.

30. The composition of claim 22, wherein said potentiating agent is an immunostimulatory agent.

31. The composition of claim 30, wherein said immunostimulatory agent is a toll-like receptor (TLR) ligand.

32. A pharmaceutical composition comprising a therapeutically effective amount of the composition of any one of claims 1-31 and one or more pharmaceutically acceptable carriers or excipients.

33. A method of treatment comprising administering the pharmaceutical composition of claim 32 to a subject in need thereof.

34. The method of claim 33, wherein said pharmaceutical composition is administered without an adjuvant.

35. The method of claim 33 or 34, wherein said pharmaceutical composition is administered before or after administration of a potentiating agent.

36. The method of any one of claims 33-35, wherein said pharmaceutical composition is administered substantially simultaneously with a potentiating agent.

37. The method of claim 35 or 36, wherein said potentiating agent is a small molecule.

38. The method of claim 37, wherein said small molecule is an anti-cancer agent.

39. The method of claim 38, wherein said anti-cancer agent is a tyrosine kinase inhibitor.

40. The method of claim 39, wherein said tyrosine kinase inhibitor is Crizotinib.

41. The method of claim 39, wherein said tyrosine kinase inhibitor is Ceritinib.

42. The method of any one of claims 33-41, wherein said subject has cancer.

43. The method of claim 42, wherein said cancer is a solid tumor cancer.

44. The method of claim 42, wherein said cancer is an ALK⁺ cancer.

45. The method of any one of claims 42-44, wherein said cancer is anaplastic large cell lymphoma, non-small-cell lung cancer, neuroblastoma, rhabdomyosarcoma, neuroectodermal cancer, glioblastoma, breast carcinoma, melanoma, inflammatory myofibroblastic tumor, soft tissue tumor, ALK expressing lymphoma, or ALK expressing lung, colon, or prostate carcinoma.

46. The method of any one of claims 42-44, wherein said cancer is selected from bladder cancer, pancreatic cancer, lung cancer, liver cancer, ovarian cancer, colon cancer, stomach cancer, breast cancer, prostate cancer, renal cancer, testicular cancer, thyroid cancer, uterine cancer, rectal cancer, a cancer of the respiratory system, a cancer of the urinary system, oral cavity cancer, skin cancer, leukemia, sarcoma, carcinoma, basal cell carcinoma, non-Hodgkin's lymphoma, acute myeloid leukemia (AML), chronic lymphocytic leukemia (CLL), B-cells chronic lymphocytic leukemia (B-CLL), multiple myeloma (MM), erythroleukemia, renal cell carcinoma, astrocytoma, oligoastrocytoma, biliary tract cancer, choriocarcinoma, CNS cancer, larynx cancer, small cell lung cancer, adenocarcinoma, giant (or oat) cell carcinoma, and squamous cell carcinoma.

47. The method of any one of claims 33-43, wherein said pharmaceutical composition is administered before or after surgery to remove at least some of a solid tumor in said solid tumor cancer.

48. The method of any one of claims 33-47, wherein said subject is a mammal.

49. The method of claim 48, wherein said mammal is a human.
